(19) United States
(12) Patent Application Publication
(10) Pub. No.: US 2017/0369848 A1
(43) Pub. Date: Dec. 28, 2017

(54) ENGINEERING MESENCHYMAL STEM CELLS USING HOMOLOGOUS RECOMBINATION

(71) Applicant: Q Therapeutics, Inc., Salt Lake City, UT (US)

(72) Inventors: Mahendra S. RAO, Timonium, MD (US); Xianmin ZENG, Novato, CA (US)

(21) Appl. No.: 15/525,403

(22) PCT Filed: Nov. 10, 2015
(86) PCT No.: PCT/US2015/059833

§ 371 Date: May 9, 2017

Related U.S. Application Data
(60) Provisional application No. 62/078,000, filed on Nov. 11, 2014.

(51) Int. Cl. C12N 5/0775; C12N 15/79; C12N 9/22; C12N 5/00

(52) U.S. Cl. CPC C12N 5/0663; C12N 9/22; C12N 15/79; C07K 2319/80; C12N 5/00; C12N 2510/00

(57) ABSTRACT

Methods are provided herein for genetically modifying a mesenchymal stem cell, differentiating these cells and using these cells in screening and in treating diseases and disorders.

This patent application claims the benefit of priority from U.S. Provisional Patent Application Ser. No. 62/078,000 filed Nov. 11, 2014, the teachings of which are herein incorporated by reference in their entirety.

### FIELD OF THE INVENTION

This invention relates to the field of mesenchymal stem cells (MSCs), specifically to methods and compositions for modifying the genome and/or genomic DNA of mesenchymal stem cells.

### BACKGROUND

Stem cells can be classified as embryonic or adult, depending on their tissue of origin. The role of adult stem cells is to sustain an established repertoire of mature cell types in essentially steady-state numbers over the lifetime of the organism. Although adult tissues with a high turnover rate, such as blood, skin, and intestinal epithelium, are maintained by tissue-specific stem cells, the stem cells themselves rarely divide. However, in certain situations, such as during tissue repair after injury or following transplantation, stem cell division may become more frequent. The prototypic example of adult stem cells, the hematopoietic stem cells, has already been demonstrated to be of utility in gene therapy. Although they are relatively rare in the human body, these cells can be readily isolated from bone marrow or after mobilization into peripheral blood. Specific surface markers allow the identification and enrichment of hematopoietic stem cells from a mixed population of bone marrow or peripheral blood cells.

After in vitro manipulation, these cells may be retransplanted into patients by injection into the bloodstream, where they travel in response to endogenous cues to the place in the bone marrow in which they are functionally active. Hematopoietic stem cells that have been explanted, in vitro manipulated, and retransplanted into the same patient (autologous transplantation) or a different patient (allogeneic transplantation) retain the ability to contribute to all mature blood cell types of the recipient for an extended period of time.

Another adult bone marrow-derived stem cell type with potential use as a vehicle for gene transfer is the mesenchymal stem cell, which has the ability to form cartilage, bone, adipose tissue, and marrow stroma. Related stem cell types have also been described, such as: the multipotent adult progenitor cell, which has been isolated from bone marrow and can differentiate into multiple lineages, which can include neurons, hepatocytes, endothelial cells, and other cell types; the mesenchymal progenitor cells described by Mesoblast, Ltd; and multipotent cells sourced from placental tissue described by Celgene, Inc. Other adult stem cells have been identified, such as those in the central nervous system and heart, but these are less well characterized and not as easily accessible.

A traditional method for introducing a therapeutic gene into hematopoietic stem cells from bone marrow or peripheral blood involves the use of a vector derived from a certain class of virus, called a retrovirus. One type of retroviral vector was initially employed to show proof-of-principle. Since most adult stem cells divide at a relatively slow rate, efficiency was rather low. Vectors derived from other types of retroviruses (lentiviruses) and adenoviruses have the potential to overcome this limitation, since they also target non-dividing cells. The major drawback of these methods is that the therapeutic gene frequently integrates more or less randomly into the chromosomes of the target cell. In principle, this is dangerous, because the gene therapy vector can potentially modify the activity of neighboring genes (positively or negatively) in close proximity to the insertion site or even inactivate host genes by integrating into them. These phenomena are referred to as “insertional mutagenesis”. In extreme cases, such as in the X-linked SCID gene therapy trials, these mutations contribute to the malignant transformation of the targeted cells, ultimately resulting in cancer.

Safe-harbor loci, which allow for robust expression of a transgene integrated into the genome of a cell, provide a defined insertion cite for exogenous DNA such as mini-gene and reporter cassettes. For example, PPP1R12C/AAVS1 and hRosa26 safe harbors have been used in genome engineering of human pluripotent stem cells by conventional or nuclease-enhanced gene targeting (Irion, S. et al. Nature Biotechnology 25, 1477-1482 (2007) and Zou, J et al., Blood 117, 5561-5572 (2011)). While Zinc Finger Nuclease (ZFN), transcription activator-like effector nuclease (TALEN), and clustered regularly interspaced short palindromic repeat (CRISPR) RNA-guided Cas nuclease (CRISPR/Cas) have been used to show efficient gene editing in pluripotent stem cells (Hockmeyer, D. et al., Nature Biotechnology 29, 731-734 (2011); Mali, P. et al., Science 339, 823-826 (2013); Zou, J. et al., Cell Stem Cell 5, 97-110 (2009)), one-step modification of multiple loci in stem cells was only recently demonstrated in mouse embryonic stem cells (ESCs) and embryo by non-homologous end-joining (NHEJ) or homology-directed repair (HDR) (Wang, H. et al., Cell 153, 910-918 (2013) and Yang, H, et al., Cell 154, 1370-1379 (2013)). To date, multiplexed knock-in or transfer of large DNA fragments has not been reported in human pluripotent or multi-potent stem cells, although engineered human stem cells are highly valuable for multi-lineage labeling, drug screening, and gene therapy.

While gene engineering and homologous recombination has been possible in pluripotent stem cells, it has been difficult in adult cells. One reason for this has been the low efficiency of homologous recombination and the limited replication potential of adult stem and progenitor cells. Many attempts to develop such technologies have been tried but homologous recombination has been limited to immortalized lines and spontaneously immortal cells which have unlimited replication potential.

Another limitation in using adult stem cells is that it is relatively difficult to maintain the stem cell state during ex vivo manipulations. Under current suboptimal conditions, adult stem cells tend to lose their stem cell properties and become more specialized, giving rise to mature cell types through a process termed differentiation. Recent advances in supportive culture conditions for mouse hematopoietic stem cells may ultimately facilitate more effective use of human hematopoietic stem cells in gene therapy applications.

A third limitation is that adult stem and progenitor cells undergo senescence.

### SUMMARY OF THE INVENTION

An aspect of the present invention relates to methods for modifying the genome of a MSC.

Another aspect of the present invention relates to methods for differentiating a MSC.

Another aspect of the present invention relates to methods for treating a subject that include administering an effective amount of MSCs produced by the methods disclosed herein or cells differentiated from an MSC produced by the methods disclosed herein.

In one embodiment, a method is provided for introducing a polynucleotide of interest into a safe harbor locus in a genome of a MSC. The method includes introducing into the MSC (a) an upstream transcription activator-like effector nuclease (TALEN) comprising an upstream DNA-binding domain linked to a DNA cleavage domain, wherein the upstream DNA binding domain specifically binds to the safe-harbor locus at a site upstream of a genomic insertion site in the genome of the mesenchymal stem cell, (b) a downstream transcription activator-like effector nuclease (TALEN) comprising a downstream DNA-binding domain linked to a DNA cleavage domain, wherein the downstream DNA binding domain specifically binds to the safe-harbor locus at a site downstream of the genomic insertion site in the genome of the mesenchymal stem cell, and (c) a single or double-stranded donor polynucleotide comprising sense and/or antisense strand polynucleotide overhangs that are complementary to corresponding polynucleotide overhangs of cleaved the genomic DNA when cleaved at the genomic insertion site. The complementary overhangs facilitate homologous recombination of the donor polynucleotide with the cleaved genomic DNA, thereby introducing the polynucleotide into the genome of the MSC.

In an additional embodiment, a method is provided for inducing a MSC to differentiate into a selected mature cell type. The method includes introducing into the mesenchymal stem cell (a) an upstream transcription activator-like effector nuclease (TALEN) comprising an upstream DNA-binding binding domain linked to a DNA cleavage domain, wherein the upstream DNA binding domain specifically binds to the safe-harbor locus at a site upstream of a genomic insertion site in the genome of the mesenchymal stem cell, (b) a downstream transcription activator-like effector nuclease (TALEN) comprising a downstream DNA-binding domain linked to a DNA cleavage domain, wherein the downstream DNA binding domain specifically binds to the safe-harbor locus at a site downstream of the genomic insertion site in the genome of the MSC, and (c) a single or double-stranded donor polynucleotide comprising sense and/or antisense strand polynucleotide overhangs that are complementary to corresponding polynucleotide overhangs of cleaved the genomic DNA when cleaved at the genomic insertion site. The complementary overhangs facilitate homologous recombination of the donor polynucleotide with the cleaved genomic DNA, thereby introducing the donor polynucleotide into the genome of the MSC. The donor polynucleotide encodes one or more factors sufficient to differentiate the MSC into a selected mature cell type.

In another embodiment, a method is provided for treating a disease or disorder in a subject. The method includes selecting a subject with a selected disease or disorder and generating a MSC producing a polypeptide useful in treatment of the disease or disorder. The mesenchymal stem cell is obtained by introducing into the mesenchymal stem cell (a) an upstream transcription activator-like effector nuclease (TALEN) comprising an upstream DNA-binding domain linked to a DNA cleavage domain, wherein the upstream DNA binding domain specifically binds to the safe-harbor locus at a site upstream of a genomic insertion site in the genome of the mesenchymal stem cell, (b) a downstream transcription activator-like effector nuclease (TALEN) comprising a downstream DNA-binding domain linked to a DNA cleavage domain, wherein the downstream DNA binding domain specifically binds to the safe-harbor locus at a site downstream of the genomic insertion site in the genome of the mesenchymal stem cell, and optionally (c) a single or double-stranded donor polynucleotide comprising sense and/or antisense strand polynucleotide overhangs that are complementary to corresponding polynucleotide overhangs of cleaved genomic DNA when cleaved at the genomic insertion site, wherein the complementary overhangs facilitate homologous recombination of the donor polynucleotide with the cleaved genomic DNA, thereby introducing the donor polynucleotide into the genome of the MSC. A therapeutically effective amount of the MSC, or one or more cells differentiated from the MSC, can be administered to the subject, thereby treating the disease or disorder.

In one nonlimiting embodiment, the disease or disorder is an inflammatory or immune, a neurological, a cancer or a cardiovascular disease or disorder.

In one nonlimiting embodiment, the disease or disorder relates to absence of a protein such as an enzyme in, for example, lysosomal storage disorders, a growth factor useful, for example, in enhancing bone regrowth and/or accelerating ulcer repair or limb ischemia, or a cytokine useful in alleviating pain relating to an immune disorder such as rheumatoid arthritis.

In another nonlimiting embodiment, the MSC produces an antibody, useful in treating a disease or disorder wherein antibody treatment is warranted.

In a further embodiment, a method is provided for modifying the genomic DNA of a MSC. The method includes introducing into the cell (a) an upstream transcription activator-like effector nuclease (TALEN) comprising an upstream DNA-binding domain linked to a DNA cleavage domain, wherein the upstream DNA binding domain specifically binds to a site upstream of a genomic sequence of interest, and (b) a downstream transcription activator-like effector nuclease (TALEN) comprising a downstream DNA-binding domain linked to a DNA cleavage domain. The downstream DNA binding domain specifically binds to a site downstream of a genomic sequence of interest, and the transcription activator-like effector nucleases cleave the genomic DNA and excise the genomic sequence of interest, thereby modifying the genomic DNA of the MSC.

In another embodiment, a method is provided for treating a disorder, such as a disease resulting from dominant mutations. The method includes selecting a subject with a disease resulting from dominant mutations and generating a MSC producing a polypeptide of interest. The mesenchymal stem cell is obtained by introducing into the cell (a) an upstream transcription activator-like effector nuclease (TALEN) comprising an upstream DNA-binding domain linked to a DNA cleavage domain, wherein the upstream DNA binding domain specifically binds to a site upstream of a genomic sequence of interest, and (b) a downstream transcription activator-like effector nuclease (TALEN) comprising a downstream DNA-binding domain linked to a DNA cleavage domain. The downstream DNA binding domain specifically binds to a site downstream of a genomic sequence of interest, and the transcription activator-like effector nucleases cleave the genomic DNA and excise the genomic sequence of interest, thereby modifying the genomic DNA of the MSC.

The foregoing and other features and advantages of the invention will become more apparent from the following detailed description of a several embodiments which proceeds with reference to the accompanying figures.

### BRIEF DESCRIPTION OF THE FIGURES

FIG. 1. AAVS-copGFP donor vector targeting AAVS safe harbor site on Chr. 19. Experimental strategy of generating AAVS1-copGFP lines. The solid black triangles represent the loxP sites and the triangles filled with diagonal lines represent Lox sites for RMCE. Testing primer sets for 5′ (Left arm integration test), 3′ (Right arm integration test) and “ORF” (WT ORF test) are also illustrated.

FIG. 2A-2D Generation of MSC line stably expressing AAVS-copGFP. The process of generating a stable AAVS-copGFP MSC line is illustrated in the flowchart (FIG. 2A). One day after nucleofection with AAVS-copGFP, ˜60% of MSCs were observed to contain the transient green plasmid (FIG. 2B). After two weeks of drug selection, the majority (>98%) of MSCs were stably expressing green fluorescence (FIG. 2C). The successful integration of the plasmid in this mixed cell population was confirmed by junction PCR (FIG. 2D).

### SEQUENCE LISTING

The nucleic and amino acid sequences disclosed herein use standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. Sequence names for SEQ ID NOs 1-21 as set forth in the Sequence Listing provided herewith are as follows:

| SEQ ID | |
|---|---|
| NO: | Sequence name |
| 1 | Upstream CLYBL target |
| 2 | Upstream CLYBL TALE binding domain |
| 3 | Downstream CLYBL target |
| 4 | Downstream CLYBL TALE binding domain |
| 5 | Upstream TALEN - Includes Δ152 N-terminus |
| | and +63 C-terminus |
| 6 | Downstream TALEN - Includes Δ152 N- |
| | terminus and +63 C-terminus |
| 7 | Upstream CLYBL TALE binding domain |
| 8 | Upstream TALEN - Includes Δ152 N-terminus |
| | and +63 C-terminus |
| 9 | pZT-C13-L |
| 10 | Downstream CLYBL TALE binding domain |
| 11 | Downstream TALEN - Includes Δ152 N- |
| | terminus and +63 C-terminus |
| 12 | pZT-C13-R |
| 13 | FokI Nuclease |
| 14 | FokI Nuclease |
| 15 | Nuclear localization signal |
| 16 | Nuclear localization signal |
| 17 | FLAG tag |
| 18 | FLAG tag |
| 19 | CLYBL target region |
| 20 | Primer |
| 21 | Primer |

### DETAILED DESCRIPTION OF THE INVENTION

The present invention provides a strategy for targeting a safe harbor locus in mesenchymal stem cells (MSCs) to modify the genome of the MSC. In this strategy, the inserted gene is not silenced due to incorporation of insulators. Further, the same site can be targeted repeatedly using the gene editing tools identified herein for this locus. In the present invention, unique TALENS have been designed which successfully target mesenchymal stem cells. It is expected that this strategy can be used to target any stem cells and/or progenitor cells that have the same or similar replicative potential to MSCs.

Methods are available for designing TALENs (Bogdanove and Voytas, Science. 2011 Sep. 30; 333(6051):1843-6. doi: 10.1126/science.1204094), and TALEN-mediated gene targeting is as effective as ZFNs in human embryonic stem cells (hESCs) and iPSCs (Hockenmeyer et al., Nat Biotechnol 29: 731-734). Genomic editing with TALENs and ZFNs capitalizes on the cell's ability to undergo homology directed repair (HDR), following an induced and targeted double-stranded DNA break (DSB). During this time a donor DNA template can be provided to the cell to insert new transgene or delete DNA sequences at the site of DSB (Cheng et al., Genes Cells. 2012 June; 17(6):431-8. doi: 10.1111/j.1365-2443.2012.01599.x. Epub 2012 Apr. 4).

### Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). In order to facilitate review of the various embodiments of this disclosure, the following explanations of specific terms are provided:

Animal: Living multi-cellular vertebrate organisms, a category that includes, for example, mammals and birds. The term mammal includes both human and non-human mammals. Similarly, the term “subject” includes both human and veterinary subjects.

Cell Culture: Cells grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Differentiation: The process whereby relatively unspecialized cells (e.g., embryonic cells or stem cells) acquire specialized structural and/or functional features characteristic of mature cells. Similarly, “differentiate” refers to this process. Typically, during differentiation, cellular structure alters and tissue-specific proteins and properties appear.

Differentiation medium: A synthetic set of culture conditions with the nutrients necessary to support the growth or survival of microorganisms or culture cells, and which allows the differentiation of cells, such as mesenchymal stem cells.

Donor polynucleotide: A polynucleotide that is capable of specifically inserting into a genomic locus.

Downstream: A relative position on a polynucleotide, wherein the “downstream” position is closer to the 3′ end of the polynucleotide than the reference point. In the instance of a double-stranded polynucleotide, the orientation of 5′ and 3′ ends are based on the sense strand, as opposed to the antisense strand.

Embryonic Stem (ES) Cells: Pluripotent cells isolated from the inner cell mass of the developing blastocyst, or the progeny of these cells. “ES cells” can be derived from any organism. ES cells can be derived from mammals, including mice, rats, rabbits, guinea pigs, goats, pigs, cows, monkeys and humans. In specific, non-limiting examples, the cells are human or murine. Without being bound by theory, ES cells can generate a variety of the cells present in the body (bone, muscle, brain cells, etc.), provided they are exposed to conditions conducive to developing these cell types. Methods for producing murine ES cells can be found in U.S. Pat. No. 5,670,372, which is herein incorporated by reference. Methods for producing human ES cells can be found in U.S. Pat. No. 6,090,622, WO 00/70021 and WO 00/27995, which are herein incorporated by reference.

Effective amount or Therapeutically effective amount: The amount of agent, such a cell, for example MSCs, that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

Exogenous: Not normally present in a cell, but can be introduced by genetic, biochemical or other methods. Exogenous nucleic acids include DNA and RNA, which can be single or double-stranded; linear, branched or circular; and can be of any length. By contrast, an “endogenous” molecule is one that is normally present in a particular cell at a particular developmental stage under particular environmental conditions.

Expand: A process by which the number or amount of cells in a culture is increased due to cell division. Similarly, the terms “expansion” or “expanded” refers to this process. The terms “proliferate,” “proliferation” or “proliferated” may be used interchangeably with the words “expand,” “expansion” or “expanded.” Typically, during an expansion phase, the cells do not differentiate to form mature cells.

Expansion medium: A synthetic set of culture conditions suitable for the expansion of cells, such as mesenchymal stem cells. Tissue culture media generally include a carbon source, a nitrogen source and a buffer to maintain pH. In one embodiment, a medium contains a minimal essential media, such as DMEM, supplemented with various nutrients to enhance mesenchymal stem cell growth. Additionally, the minimal essential media may be supplemented with additives such as horse, calf or fetal bovine serum.

FokI nuclease: A nonspecific DNA nuclease that occurs naturally in Flavobacterium okeanokoites. The term includes fragments of the FokI nuclease protein that retain nuclease activity that are, or may be, fused to a DNA-binding polypeptide.

Genomic insertion site: A site of the genome that is targeted for, or has undergone, insertion of an exogenous polynucleotide.

Growth factor: A substance that promotes cell growth, survival, and/or differentiation. Growth factors include molecules that function as growth stimulators (mitogens), molecules that function as growth inhibitors (e.g. negative growth factors) factors that stimulate cell migration, factors that function as chemotactic agents or inhibit cell migration or invasion of tumor cells, factors that modulate differentiated functions of cells, factors involved in apoptosis, or factors that promote survival of cells without influencing growth and differentiation. Examples of growth factors are bFGF, epidermal growth factor (EGF), CNTF, HGF, nerve growth factor (NGF), and actvin-A.

Heterologous: A heterologous sequence is a sequence that is not normally (i.e. in the wild-type sequence) found adjacent to a second sequence. In one embodiment, the sequence is from a different genetic source, such as a virus or organism, than the second sequence.

Induced pluripotent stem cell” (“iPS” cell or “iPSC”): A pluripotent stem cell artificially derived from a non-pluripotent cell, typically an adult somatic cell, by recombinant expression of specific factors in the non-pluripotent pluripotent cell. Factors that may be used to for iPSCs include, but are not limited to, one or more of Oct-3/4, certain members of the Sox gene family (Sox1, Sox2, Sox3, and Sox15, Klf family members (Klf1, Klf2, Klf4, and Klf5), factors of the Myc family (c-myc, L-myc, and N-myc), Nanog, and LIN28, as defined by current knowledge in the art. Other factors or methods useful for creating iPSCs are also known in the art and are considered to produce cells that fall within the scope of this definition.

Isolated: An “isolated” biological component (such as a nucleic acid, peptide or cell) has been substantially separated, produced apart from, or purified away from other biological components or cells of the organism in which the component naturally occurs, i.e., other chromosomal and extrachromosomal DNA and RNA, cells and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Lineage-specific: Characteristics of a cell that indicate the cell will become one of a limited number of related cell types or a particular cell type, such as a differentiated cell or a cell undergoing the process of differentiation into a specific cell type or a mature cell type.

Mesenchymal Stem Cell (MSC): Also referred to as multipotent stromal cells and meant to be inclusive not only of MSCs but also of cells with replicative potential similar thereto that can differentiate into a variety of cell types. Additional examples of cells meant to be encompassed herein by the terms MSC and/or mesenchymal stem cells include, but are not limited to, mesenchymal precursor cells or MPCs, mesenchymal progenitor cells such as described by Mesoblast, Ltd., and other adult-derived stem cells such as MULTISTEM (Athersys, Inc.). While these multipotent stem cells are traditionally found in the bone marrow, they can also be isolated from other tissues including, but not limited to, cord blood, peripheral blood, fallopian tube, fetal liver and lung, placenta and fat. MSCs and other adult stem cells which can be used in accordance with the present invention, differentiate to form cells and/or tissues including, but not limited, adipocytes, cartilage, bone, tendons, muscle, and skin as well as myocytes, neurons and glia.

Modulate: A change in the content of genomic DNA gene. Modulation can include, but is not limited to, gene activation, gene repression, gene deletion, polynucleotide insertion, and polynucleotide excision.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Pharmaceutical agent or “drug”: A chemical compound or composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Polypeptide: Three or more covalently attached amino acids. The term encompasses proteins, protein fragments, and protein domains. A “DNA-binding” polypeptide is a polypeptide with the ability to specifically bind DNA.

The term “polypeptide” is specifically intended to cover naturally occurring proteins, as well as those which are recombinantly or synthetically produced. The term “functional fragments of a polypeptide” refers to all fragments of a polypeptide that retain an activity of the polypeptide. Biologically functional fragments, for example, can vary in size from a polypeptide fragment as small as an epitope capable of binding an antibody molecule to a large polypeptide capable of participating in the characteristic induction or programming of phenotypic changes within a cell. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions replace one amino acid with another amino acid that is similar in size, hydrophobicity, etc. Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Variations in the cDNA sequence that result in amino acid changes, whether conservative or not, should be minimized in order to preserve the functional and immunologic identity of the encoded protein. The immunologic identity of the protein may be assessed by determining whether it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may, for example, be 80%, 90% or even 95% or 98% identical to the native amino acid sequence.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one coded for by a recombinant nucleic acid molecule.

Recombination: A process of exchange of genetic information between two polynucleotides. “Homologous recombination (HR)” refers to the specialized form of an exchange that takes place, for example, during repair of double-strand breaks in cells. Nucleotide sequence homology is utilized in recombination, for example using a “donor” molecule to template repair of a “target” molecule (i.e., the one that experienced the double-strand break), and is variously known as “non-crossover gene conversion” or “short tract gene conversion,” because it leads to the transfer of genetic information from the donor to the target.

Safe harbor: A locus in the genome where a polynucleotide may be inserted without causing deleterious effects to the host cell. Examples of safe harbor loci known to exist within mammalian cells may be found within the AAVS1 gene, the CYBL gene, and the CCR5 gene.

Selectable marker: A gene introduced into a cell, such mammalian cells in culture, for example a MSC, that confers a trait suitable for artificial selection from cells that do not possess the gene.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a FGF polypeptide will possess a relatively high degree of sequence identity when aligned using standard methods.

Methods of alignment of sequences for comparison are well known in the art. Various programs and alignment algorithms are described in Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul, et al., Nature Genet., 6:119, 1994 presents a detailed consideration of sequence alignment methods and homology calculations.

The NCBI Basic Local Alignment Search Tool (BLAST) (Altschul, et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a FGF polypeptide are typically characterized by possession of at least about 75%, for example at least about 80%, sequence identity counted over the full length alignment with the amino acid sequence of the factor using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Specific binding: A sequence-specific, non-covalent interaction between macromolecules (e.g., between a polypeptide and a polynucleotide). Not all components of a binding interaction need be sequence-specific (e.g., contacts with phosphate residues in a DNA backbone), as long as the interaction as a whole is sequence-specific. The term should not be construed to indicate that a macromolecule described as participating in specific binding, or as being specific for another given macromolecule, cannot bind to another macromolecule, but rather that the specific nature of the interaction is significantly favored over a nonspecific or random binding. Such “specific binding” interactions are generally characterized by a dissociation constant (K_{d}) of 10⁻⁶ M⁻¹ or lower.

Subject: Human and non-human animals, including all vertebrates, such as mammals and non-mammals, such as non-human primates, mice, rabbits, sheep, dogs, cats, horses, cows, chickens, amphibians, and reptiles. In many embodiments of the described methods, the subject is a human.

Synapse: Highly specialized intercellular junctions between neurons and between neurons and effector cells across which a nerve impulse is conducted (synaptically active). Generally, the nerve impulse is conducted by the release from one neuron (presynaptic neuron) of a chemical transmitter (such as dopamine or serotonin) which diffuses across the narrow intercellular space to the other neuron or effector cell (post-synaptic neuron). Generally neurotransmitters mediate their effects by interacting with specific receptors incorporated in the post-synaptic cell.

“Synaptically active” refers to cells (e.g., differentiated neurons) which receive and transmit action potentials characteristic of mature neurons.

Transduced, Transformed and Transfected: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed, Gene Therapeutics, Birkhauser, Boston, USA, 1994). In the case of infection by retroviruses, the infecting retrovirus particles are absorbed by the target cells, resulting in reverse transcription of the retroviral RNA genome and integration of the resulting provirus into the cellular DNA. Methods for the introduction of genes into cells are known (e.g. see U.S. Pat. No. 6,110,743, herein incorporated by reference). These methods can be used to transduce a MSC or a cell produced by the methods described herein.

Genetic modification of the target cell is an indicium of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene.

Treating, Treatment, and Therapy: Any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

Upstream: A relative position on a polynucleotide, wherein the “upstream” position is closer to the 5′ end of the polynucleotide than the reference point. In the instance of a double-stranded polynucleotide, the orientation of 5′ and 3′ ends are based on the sense strand, as opposed to the antisense strand.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Zinc finger DNA binding domain: A polypeptide domain that binds DNA in a sequence-specific manner through one or more zinc fingers, which are regions of amino acid sequence within the binding domain whose structure is stabilized through coordination of a zinc ion.

Zinc finger binding domains, for example the recognition helix of a zinc finger, can be “engineered” to bind to a predetermined nucleotide sequence. Rational criteria for design of zinc finger binding domains include application of substitution rules and computerized algorithms for processing information in a database storing information of existing ZFP designs and binding data, see for example U.S. Pat. No. 5,789,538; U.S. Pat. No. 5,925,523; U.S. Pat. No. 6,007,988; U.S. Pat. No. 6,013,453; U.S. Pat. No. 6,140,081; U.S. Pat. No. 6,200,759; U.S. Pat. No. 6,453,242; and U.S. Pat. No. 6,534,261; and PCT Publication Nos. WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084 and WO 03/016496.

The term “about” as used herein when referring to a measurable value such as an amount, a temporal duration, and the like, is meant to encompass variations of up to ±10% from the specified value. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed subject matter. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, “A or B” is intended to include “A,” “B,” and “both A and B,” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Compositions for Targeting MSCs

Disclosed below are compositions that can be used to genetically modify MSCs and other stem cells and/or progenitor cells that have the same or similar replicative potential. These compositions can be used in any of the methods disclosed herein.

### DNA-Binding Polypeptides

The recombinant polynucleotide-binding polypeptides of use in the methods disclosed herein can occur in a variety of forms. In some embodiments, the recombinant polynucleotide-binding polypeptide is a recombinant DNA-binding polypeptide that specifically binds to a genomic target sequence in a mesenchymal stem cell. In one embodiment the targeted genomic sequence bound by the recombinant DNA-binding polypeptide falls within the sequence of SEQ ID NO: 19, or its corresponding antisense sequence. In another embodiment the targeted sequence bound by the recombinant DNA-binding polypeptide in the genome of the mesenchymal stem cell includes the sequence of SEQ ID NO: 1. In yet another embodiment, the targeted sequence bound by the recombinant DNA-binding polypeptide is the sequence of SEQ ID NO: 1. Alternatively, the targeted sequence bound by the recombinant DNA-binding polypeptide may include a sequence that is antisense, or complementary, to the sequence of SEQ ID NO: 1. In one embodiment, the targeted sequence bound by the recombinant DNA-binding polypeptide is a sequence that is antisense, or complementary, to the sequence of SEQ ID NO: 1. In another embodiment the targeted sequence bound by the recombinant DNA-binding polypeptide includes the sequence of SEQ ID NO: 3. In a further embodiment, the targeted sequence bound by the recombinant DNA-binding polypeptide is the sequence of SEQ ID NO: 3. Alternatively, the targeted sequence bound by the recombinant DNA-binding polypeptide can include a sequence that is antisense, or complementary, to the sequence of SEQ ID NO: 3. In one embodiment, the targeted sequence bound by the recombinant DNA-binding polypeptide is a sequence that is antisense, or complementary, to the sequence of SEQ ID NO: 3.

In some embodiments the described recombinant DNA-binding polypeptide includes a zinc-finger domain or a transcription activator-like effector (TALE) domain, or a polypeptide fragment thereof that retains the DNA binding function of the TALE domain or the zinc-finger domain. Furthermore, the recombinant DNA-binding polypeptide may also be combined with a polypeptide having nuclease activity, such as a zinc-finger domain or a transcription activator-like effector (TALE) domain fused to a nuclease protein, or a fragment thereof. Exemplary nucleases include, but are not limited to, S1 nuclease, mung bean nuclease, pancreatic DNAase I, micrococcal nuclease, and yeast HO endonuclease (see also Linn et al. (eds.) Nucleases, Cold Spring Harbor Laboratory Press, 1993).

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at nine nucleotides from its recognition site on one strand and 13 nucleotides from its recognition site on the other (see, for example, U.S. Pat. Nos. 5,356,802; 5,436,150 and 5,487,994; Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982). Thus, in one embodiment, a nuclease domain from at least one Type IIS restriction enzyme is utilized. An exemplary Type IIS restriction enzyme, whose cleavage domain is separable from the binding domain, is Fok1. This particular enzyme is active as a dimer. See Bitinaite et al. (1998) Proc. Natl. Acad. Sci. USA 95: 10,570-10,575. Additional forms of FokI nuclease are set forth in U.S. Published Patent Application No. 20110027235, which is incorporated herein by reference.

In some embodiments the polypeptide having nuclease activity that is fused with the recombinant DNA-binding polypeptide is the FokI nuclease, or a derivative or fragment thereof that retains the nuclease activity. In some embodiments, the Fok1 nuclease is at least about 80%, at least about 85%, at least about 90%, at least about 95%, at least about 96%, at least about 97%, at least about 98%, at least about 99%, or about 100% identical to SEQ ID NO: 13.

In the case of a recombinant DNA-binding polypeptide produced from a TALE domain, fusion with a polypeptide having nuclease activity forms a transcription activator-like effector nuclease (TALEN). Some of the TALEN embodiments described herein are designed to specifically target a genomic sequence that falls within the sequence of SEQ ID NO: 19, or its corresponding antisense sequence, such as, for example, the sequence of SEQ ID NO: 1 or 3. In one embodiment the targeted sequence bound by a described TALE domain includes the sequence of SEQ ID NO: 1. In one embodiment, the targeted sequence bound by a described TALE domain is the sequence of SEQ ID NO: 1. Alternatively, the targeted sequence bound by a described TALE domain may include a sequence that is antisense, or complementary, to the sequence of SEQ ID NO: 1. In one embodiment, the targeted sequence bound by a described TALE domain is a sequence that is antisense, or complementary, to the sequence of SEQ ID NO: 1. In another embodiment the targeted sequence bound by a described TALE domain includes the sequence of SEQ ID NO: 3. In one embodiment, the targeted sequence bound by a described TALE domain is the sequence of SEQ ID NO: 3. Alternatively, the targeted sequence bound by a described TALE domain may include a sequence that is antisense, or complementary, to the sequence of SEQ ID NO: 3. In one embodiment, the targeted sequence bound by a described TALE domain is a sequence that is antisense, or complementary, to the sequence of SEQ ID NO: 3.

The TALE domains of use in the methods disclosed herein can be linked to a polypeptide having nuclease activity to form a TALEN, which can be used to cleave DNA at a specific location of interest. In one embodiment the targeted sequence bound by a described TALEN includes the sequence of SEQ ID NO: 1. In one embodiment, the targeted sequence bound by a described TALEN is the sequence of SEQ ID NO: 1. Alternatively, the targeted sequence bound by a described TALEN may include a sequence that is antisense, or complementary, to the sequence of SEQ ID NO: 1. In one embodiment, the targeted sequence bound by a described TALEN is a sequence that is antisense, or complementary, to the sequence of SEQ ID NO: 1. In another embodiment the targeted sequence bound by a described TALEN includes the sequence of SEQ ID NO: 3. In one embodiment, the targeted sequence bound by a described TALEN is the sequence of SEQ ID NO: 3. Alternatively, the targeted sequence bound by a described TALEN may include a sequence that is antisense, or complementary, to the sequence of SEQ ID NO: 3. In one embodiment, the targeted sequence bound by a described TALEN is a sequence that is antisense, or complementary, to the sequence of SEQ ID NO: 3.

For the methods disclosed herein, the recombinant DNA-binding polypeptide may also be combined with a polypeptide having nuclease activity, such as a zinc-finger domain or a transcription activator-like effector (TALE) domain fused to a nuclease protein, or a fragment thereof. In some embodiments the polypeptide having nuclease activity that is fused with the recombinant DNA-binding polypeptide is the fokI nuclease, or a derivative or fragment thereof that retains the nuclease activity. In the case of a recombinant DNA-binding polypeptide produced from a TALE domain, fusion with a polypeptide having nuclease activity forms a transcription activator-like effector nuclease (TALEN).

Some of the TALEN embodiments of use in the disclosed methods are designed to specifically target a genomic sequence that falls within the sequence of SEQ ID NO: 19, or its corresponding antisense sequence, such as, for example, the sequence of SEQ ID NO: 1 or 3. In one embodiment the TALE domain includes the amino acid sequence of SEQ ID NO: 7. In another embodiment the TALE domain includes an amino acid sequence of SEQ ID NO: 10. In further embodiments a TALE domain is fused to a polypeptide having nuclease activity to form a TALEN. One TALEN of use in the methods disclosed herein is a TALE domain that includes the amino acid sequence of SEQ ID NO: 7 incorporated into a polypeptide having nuclease activity. In one such embodiment, the amino acid sequence of SEQ ID NO: 7 is incorporated into a polypeptide that also includes a fokI nuclease, or a fragment thereof. For example, the amino acid sequence of SEQ ID NO: 7 may be incorporated into a polypeptide that also includes the amino acid sequence of SEQ ID NO: 13. One embodiment of a polypeptide where the amino acid sequence of SEQ ID NO: 7 is incorporated with the amino acid sequence of SEQ ID NO: 13, is the polypeptide of SEQ ID NO: 8. One TALEN of use in the methods disclosed herein is a TALE domain that includes the amino acid sequence of SEQ ID NO: 10 incorporated into a polypeptide having nuclease activity. In one such embodiment, the amino acid sequence of SEQ ID NO: 10 is incorporated into a polypeptide that also includes a fokI nuclease, or a fragment thereof that retains nuclease activity. For example, the amino acid sequence of SEQ ID NO: 10 may be incorporated into a polypeptide that also includes the amino acid sequence of SEQ ID NO: 13. One embodiment of a polypeptide where the amino acid sequence of SEQ ID NO: 10 is incorporated with the amino acid sequence of SEQ ID NO: 13, is the polypeptide of SEQ ID NO: 11.

The TALE constructs of use in the methods disclosed herein can be used to target specific DNA sequences, such as a genomic sequence of interest in an MSC. When coupled with a polypeptide having nuclease activity to form a TALEN, these constructs can be used to target a specific polynucleotide of interest for modification in the genome of the MSC. In one embodiment the described TALE domain includes the amino acid sequence of SEQ ID NO: 7 which can target the sequence of SEQ ID NO: 1 specifically. In another embodiment the TALE domain includes an amino acid sequence of SEQ ID NO: 10 which can target the sequence of SEQ ID NO: 3 specifically. In further embodiments a described TALE domain is fused to a polypeptide having nuclease activity to form a TALEN. One TALEN described herein is a TALE domain that includes the amino acid sequence of SEQ ID NO: 7 incorporated into a polypeptide having nuclease activity, which can target the sequence of SEQ ID NO: 1 specifically. In one such embodiment, the amino acid sequence of SEQ ID NO: 7 is incorporated into a polypeptide that also includes a fokI nuclease, or a fragment thereof that retains nuclease activity, and can target the sequence of SEQ ID NO: 1 specifically and mediate cleavage of a DNA sequence proximal to the segment where the polynucleotide is bound. For example, the amino acid sequence of SEQ ID NO: 7 may be incorporated into a polypeptide that also includes the amino acid sequence of SEQ ID NO: 13, for specific targeting of the sequence of SEQ ID NO: 1 and cleavage of the polynucleotide sequence proximal to the binding locus. One embodiment of a polypeptide where the amino acid sequence of SEQ ID NO: 7 is incorporated with the amino acid sequence of SEQ ID NO: 13, is the polypeptide of SEQ ID NO: 8, which can specifically bind the sequence of SEQ ID NO: 1 and cleave the polynucleotide sequence proximal to the binding locus.

Another TALEN of use in the methods disclosed herein is a TALE domain that includes the amino acid sequence of SEQ ID NO: 10 incorporated into a polypeptide having nuclease activity, which can target the sequence of SEQ ID NO: 3 specifically. In one such embodiment, the amino acid sequence of SEQ ID NO: 10 is incorporated into a polypeptide that also includes a fokI nuclease, or a fragment thereof that retains nuclease activity, and can target the sequence of SEQ ID NO: 3 specifically and mediate cleavage of a DNA sequence proximal to the segment where the polynucleotide is bound. For example, the amino acid sequence of SEQ ID NO: 10 may be incorporated into a polypeptide that also includes the amino acid sequence of SEQ ID NO: 13, for specific targeting of the sequence of SEQ ID NO: 3 and cleavage of the polynucleotide sequence proximal to the binding locus. One embodiment of a polypeptide where the amino acid sequence of SEQ ID NO: 10 is incorporated with the amino acid sequence of SEQ ID NO: 13, is the polypeptide of SEQ ID NO: 11, which can specifically bind the sequence of SEQ ID NO: 3 and cleave the polynucleotide sequence proximal to the binding locus.

Modifications can be made to the described subject matter resulting in substantially similar polypeptides and constructs that carry out essentially the same functions, in substantially the same way, as the described polynucleotide-binding polypeptides and related nuclease constructs. For example, zinc-finger-based constructs, or CRISPR technology, can be used to target the loci described herein to modify a genome of a cell or chromosomal DNA. Accordingly, such variations are considered to be within the scope of the present disclosure.

### Polynucleotides and Vectors

Polynucleotides and vectors are of use in the methods disclosed herein. The polynucleotides encode the polypeptides disclosed above. In some embodiments, the polynucleotides and vectors encode recombinant DNA-binding polypeptides, zinc-finger or TALE domains, nuclease proteins or polypeptides, fusion proteins produced from the fusion of DNA-binding polypeptides and nuclease proteins or polypeptides, such as TALENs. In some embodiments the expression of the polypeptides encoded by the vectors are controlled by an inducible promoter. Suitable promoters include, but are not limited to, the doubecourtin (DCX) promoter and glial fibrillary acidic protein (GFAP). In other embodiments the expression of the polypeptides encoded by the vectors are controlled by a repressible promoter. Mesenchymal stem cells can be modified by the described vectors, for example transfected cells or cells having an expression product of the vectors.

The polypeptides described herein can be encoded by a variety of polynucleotides due to the degeneracy of the genetic code. Thus, the polynucleotides provided herein may be altered to encode the same corresponding amino acid sequences disclosed herein, as would be understood by those skilled in the art. Accordingly, the use of such varied polynucleotide sequences should be considered within the scope of the presently claimed methods. The amino acid sequence of SEQ ID NO: 7 may be encoded by a nucleotide having the sequence of SEQ ID NO: 2. The amino acid sequence of SEQ ID NO: 8 may be encoded by a nucleotide having the sequence of SEQ ID NO: 5. The amino acid sequence of SEQ ID NO: 10 may be encoded by a nucleotide having the sequence of SEQ ID NO: 4. The amino acid sequence of SEQ ID NO: 11 may be encoded by a nucleotide having the sequence of SEQ ID NO: 6. The amino acid sequence of SEQ ID NO: 13 may be encoded by a nucleotide having the sequence of SEQ ID NO: 14.

Furthermore, the vectors of use in the methods disclosed herein, that express the polynucleotides, or produce the polypeptides, may be substituted for other vectors having similar functional capabilities that would be understood by those skilled in the art having benefit of the present disclosure. In one embodiment, the polypeptide of SEQ ID NO: 8 may be produced by the polynucleotide of SEQ ID NO: 9. In another embodiment the polypeptide of SEQ ID NO: 11 may be encoded by the polynucleotide of SEQ ID NO: 12.

Provided herein are donor polynucleotides that may be inserted into the genome of a mesenchymal stem cell. In some embodiments the donor polynucleotides are double-stranded polynucleotides with sense and/or antisense strand polynucleotide overhangs that are at least partially complementary to corresponding polynucleotide overhangs of cleaved genomic DNA to facilitate insertion of the donor polynucleotide with the cleaved genomic DNA. In additional embodiments the donor polynucleotides are single-stranded polynucleotides with sense and/or antisense strand polynucleotide overhangs (portions) that are at least partially complementary to corresponding polynucleotide overhangs of cleaved genomic DNA to facilitate insertion of the donor polynucleotide with the cleaved genomic DNA. In some embodiments the donor polynucleotide may express a polypeptide once inserted into the genome of a mesenchymal cell or a cell differentiated therefrom. In some embodiments the expressed polypeptide can be a protein that can function to induce cell differentiation or maturation to proceed in a particular manner, such as toward a specific cell lineage. In some embodiments the expression of a polypeptide by the donor polynucleotide may be controlled by an inducible promoter, such as a promoter expressed in differentiated cells. In other embodiments, the expression of a polypeptide by the donor polynucleotide may be controlled by a repressible promoter. In still other embodiments the donor polynucleotide may encode more than one polypeptide, for example, the donor polynucleotide may include an expression cassette having a plurality of genes. In certain embodiments where the donor polynucleotide encodes more than one polypeptide, the donor polynucleotide may have inducible promoters to regulate the expression of certain genes and repressible promoters to regulate the expression of other genes.

### MSCs

MSCs are of use in any of the methods disclosed herein.

By “MSCs”, when used herein, it is meant to be inclusive of mesenchymal stem cells, also commonly referred to as multipotent stromal cell, as well as other adult stem cells with replicative potential similar thereto that can differentiate to form a variety of cell types and/or tissues, including but not limited, adipocytes, cartilage, bone, tendons, muscle, and skin as well as myocytes, neurons and glia. Additional examples of cells meant to be encompassed herein by the terms MSC and/or mesenchymal stem cells include, but are not limited to mesenchymal precursor cells or MPCs, mesenchymal progenitor cells such as described by Mesoblast, Ltd., and other adult-derived stem cells such as MULTISTEM (Athersys, Inc.). MSCs can be obtained from the bone marrow of a mammal, including, but not limited to, a human. These multipotent stem cells can also be isolated from other tissues including, but not limited to, cord blood, peripheral blood, fallopian tube, fetal liver and lung, placenta and fat.

MSCs are available through commercial sources such as, but not limited to, RoosterBio, Inc. (Frederick, Md.).

Standard culture media for MSCs typically contains a variety of essential components required for cell viability, including inorganic salts, carbohydrates, hormones, essential amino acids, vitamins, and the like. In some embodiments, DMEM or F-12 is used as a culture medium. Both media are commercially available (DMEM; GIBCO, Grand Island, N.Y.; F-12, GIBCO, Grand Island, N.Y.). A premixed formulation of DMEM/F-12 is also available commercially. Additional additives can be used, such as glutamine, heparin, sodium bicarbonate and/or N2 supplement (Life Technologies, Gaithersburg, Md.). The pH of the culture medium is typically between 6-8, such as about 7, for example about 7.4. Cells are typically cultured at a temperature between 30-40° C., such as between 35-38° C., such as between 35-37° C., for example at 37° C.

Also disclosed are MSCs, and cells differentiated therefrom, that have been modified to express one or more of the polynucleotides disclosed herein. The MSCs can express any of the polypeptides disclosed above. In some embodiments a MSC is modified to include a polynucleotide including the sequence of SEQ ID NO: 2. In one embodiment a MSC is modified to include a polynucleotide including the sequence of SEQ ID NO: 4. In other embodiments a MSC is modified to include a polynucleotide including the sequence of SEQ ID NO: 5. In yet other embodiments a MSC is modified to include a polynucleotide including the sequence of SEQ ID NO: 6. In yet other embodiments, a MSC is modified to include a polynucleotide including the sequence of SEQ ID NO: 9. In additional embodiments a MSC is modified to include a polynucleotide including the sequence of SEQ ID NO: 12. Polypeptides encoded by one or more of SEQ ID NOs: 2, 4, 5, and/or 6 can be expressed by a MSC.

### Methods for Engineering MSCs

Methods are provided for modifying the genome of a MSC. In some embodiments, these methods include, but are not limited to, introducing a polynucleotide of interest into a safe harbor locus in a genome of a MSC. In additional embodiments, the methods include excise of a polynucleotide of interest from a MSC. In further embodiments, the method includes introducing a mutation into a polypeptide of interest.

The disclosed methods can target any safe harbor locus, such as AAVS1, CYBL and CCR5.

In some embodiments, the safe harbor locus is AAVS1. In additional embodiments, the methods allow for integration of a DNA into an intron of the AAVS1 safe harbor locus. In one nonlimiting embodiment wherein the safe harbor locus is AAVS1, DNA is integrated at intron 1 (between exon 1 and exon 2) of the PPP1R12C gene.

In some embodiments, the safe harbor locus is CYBL. In additional embodiments, the methods allow for integration of a DNA into an intron of the CYBL safe harbor locus. In one nonlimiting embodiment wherein the safe harbor locus is CYBL, the integration site is at intron 2 of the CYCL gene.

The MSC can be any MSC of interest, as disclosed above. In some embodiments the step of introducing a first polypeptide or TALEN into a cell involves transfecting the MSC with a polynucleotide encoding the polypeptide or TALEN. In some embodiments the step of introducing a second polypeptide or TALEN into a cell involves transfecting the cell with a polynucleotide encoding the polypeptide or TALEN. In some embodiments a single vector may be used to transfect a cell with polynucleotides that encode an upstream TALEN and the nucleic acid encoding the downstream TALEN.

Methods for introducing DNA into MSCs include chemical and physical methods. Chemical methods include liposome-based gene transfer or lipofection, calcium phosphate-mediated gene transfer, DEAE-dextran transfection techniques, and polyethyleneimine (PEI)-mediated delivery. Physical methods include ballistic gene transfer, microinjection, and nucleofection (Amaxa biosystem, 2004). In some embodiments, nucleofection is used to introduce the polynucleotides disclosed herein into MSCs. In specific non-liming examples, the nucleofection involves the use of a nucleofectin D apparatus. In some embodiments, the nucleofection provides a transfection efficiency of at least about 60%, at least about 65%, at least about 70%, at least about 75%, at least about 80%, at least about 85%, at least about 90%, or at least about 95% of the transfected cells include the introduced DNA. In specific non-limiting examples, the nucleofection provides a transfection efficiency of at least about 80%, such as at least about 85%, at least about 90%, or at least about 95%, about 96%, 97%, about 98%, or about 99% of the transfected cells include the introduced DNA.

The method can include contacting the mesenchymal stem cell with the upstream TALEN, the downstream TALEN, and the polynucleotide of interest at a ratio of about 1:1:1. In additional embodiments, a ratio of about 1:2:1 or 2:1:1 or 1:1:2 is utilized. In other embodiments, 1:3:1 or 3:1:1 or 1:1:3 is utilized. In yet other embodiments a ratio of 1:4:1 or 4:1:1 or 1:4:1 is utilized. In further embodiments a ratio of 1:5:1 or 5:1:1 or 1:1:5 is utilized.

In some embodiments, the donor polynucleotide encodes an agent for inducing the proliferation of mesenchymal stem cells. In some embodiment, the donor polynucleotide encodes an agent for inducing the differentiation of mesenchymal stem cells into a selected mature cell and/or tissue including, but not limited, adipocytes, cartilage, bone, tendons, muscle, and skin as well as myocytes, neurons and glia. The agent can be a trophic agent or a growth factor. In specific non-limiting examples, the agent is nerve growth factor, insulin, fibroblast growth factor, glial derived neurotropic factor, a Notch ligand, Delta, brain derived neurotrophic factor, glial derived neurotrophic factor, bone morphogenic protein-2 or 4 (BMP-2/4), cilliarly neurotrophic factor (CNTF), heregulin-1 beta, platelet derived growth factor (PDGF)-1 or PDGF-B. In additional embodiments, the donor polynucleotide encodes a selectable marker and/or a detectable label. Suitable detectable labels include, but are not limited to, enzymes such as horse radish peroxidase and alkaline phosphatase, and fluorescent proteins, such as green fluorescent protein.

The donor polynucleotide can include a promoter operably linked to a heterologous nucleic acid, such as a nucleic acid encoding an agent of interest and/or a selectable marker and/or a detectable marker. The promoter can be constitutive or inducible. The promoter can be a lineage specific promoter, such as a promoter suitable for expression in adipocytes, cartilage, bone, tendons, muscle, and/or skin as well as myocytes, neurons and glia. In specific non-limiting examples, the promoter is a doublecourtin (DCX) or a GFAP promoter.

In some embodiments, the donor polynucleotide is a single or double-stranded donor polynucleotide with sense and/or antisense strand polynucleotide overhangs that are complementary to corresponding polynucleotide overhangs of the cleaved genomic DNA when cleaved at the genomic insertion site. In one non-limiting example, the donor polynucleotide is single stranded.

In another non-limiting example, the donor polynucleotide is double stranded with sense and/or antisense single stranded polynucleotide overhangs that are complementary to corresponding polynucleotide overhangs of the cleaved genomic DNA. The complementary overhangs facilitate homologous recombination of the donor polynucleotide with the cleaved genomic DNA, such that the polynucleotide is introduced into the genome of the cell. In some embodiments, the overhangs are at least 15 nucleotides in length, such as 20, 30, 40, 50, 60, 70, 80, 90, 100, 200, 300, 400, 500, 600, 700, 800, 900, or 1,000 base pairs. The complementarity need not be 100% complementarity. For example, the complementary overhangs can be 95%, 96%, 97%, 98%, or 99% complementary to the overhangs of the cleaved DNA. In additional embodiments, the complementary overhangs are at least 98% or at least 99% homologous to the overhangs of the cleaved DNA.

In some embodiments, the methods include inserting a donor polynucleotide into the genome of a MSC. The donor sequence can be of any length, such as between 2 and 30,000 nucleotides in length (or any integer value therebetween), such as between 50 and 5,00 nucleotides in length, for example between about 100 and 1,000 nucleotides in length (or any integer value therebetween), or about 200 and 500 nucleotides in length (or any integer value therebetween). Techniques for determining nucleic acid and amino acid sequence identity are known in the art.

In some embodiments, the methods include introducing into the mesenchymal stem cell (a) an upstream transcription activator-like effector nuclease (TALEN) comprising an upstream DNA-binding domain linked to a DNA cleavage domain, wherein the upstream DNA binding domain specifically binds to the safe-harbor locus at a site upstream of a genomic insertion site in the genome of the mesenchymal stem cell, (b) a downstream transcription activator-like effector nuclease (TALEN) comprising a downstream DNA-binding domain linked to a DNA cleavage domain, wherein the downstream DNA binding domain specifically binds to the safe-harbor locus at a site downstream of the genomic insertion site in the genome of the mesenchymal stem cell, and (c) a single or double-stranded donor polynucleotide comprising sense and/or antisense strand polynucleotide overhangs that are complementary to corresponding polynucleotide overhangs of cleaved the genomic DNA when cleaved at the genomic insertion site. The complementary overhangs facilitate homologous recombination of the donor polynucleotide with the cleaved genomic DNA, to allow introduction of the polynucleotide into the genome of the cell. These methods provide introduction of the donor polynucleotide into the genomic insertion site into the safe harbor locus in the genome of the mesenchymal stem cell. In some embodiments, the upstream TALEN binds to the sense strand of a genomic DNA locus flanking the insertion site and the downstream TALEN binds to the antisense strand of a genomic DNA locus flanking the insertion site.

In some embodiments the upstream TALEN comprises SEQ ID NO:8. In additional embodiments, the downstream TALEN comprises SEQ ID NO:11. In further embodiments, the DNA cleavage domain comprises a FokI nuclease domain, such as, but not limited to, SEQ ID NO: 13. In some embodiments, the genomic sense strand locus bound by the upstream TALEN comprises SEQ ID NO: 1. In yet other embodiments, the genomic antisense strand locus bound by the downstream TALEN comprises SEQ ID NO: 3. In further embodiments, the donor polynucleotide is inserted into both copies of the same chromosome, such as chromosome 13, for example into an intron on chromosome 13, such as intron 2 of chromosome 13 in the CYBL gene. In some embodiments, the polynucleotide is inserted into the two copies of the same chromosome.

One application is a method of modifying the genomic DNA of a MSC, by introducing into the MSC a first polypeptide with a DNA-binding domain, having the sequence of SEQ ID NO: 7 specific for a DNA sequence upstream of a genomic sequence of interest and a second polypeptide with a DNA-binding domain, having the sequence of SEQ ID NO: 10 specific for a DNA sequence downstream from the genomic sequence of interest, wherein the first and second polypeptides mediate cleavage of the genomic DNA and excise a genomic sequence of interest, thereby modifying the genomic DNA of the MSC. Another application is a method of modifying the genomic DNA of a MSC by introducing into the MSC a first polypeptide with a DNA-binding domain specific for a DNA sequence within the sequence of SEQ ID NO: 19 upstream of a genomic sequence of interest and a second polypeptide with a DNA-binding domain specific for a DNA sequence within the sequence of SEQ ID NO: 19 downstream from the genomic sequence of interest, wherein the first and second polypeptides mediate cleavage of the genomic DNA and excises a genomic sequence of interest, thereby modifying the genomic DNA of the MSC. Yet another application is a method of modifying the genomic DNA of a MSC by introducing into the MSC a first TALEN with a DNA-binding domain specific for a DNA sequence within human chromosome 13 that binds upstream of a genomic sequence of interest and a second TALEN with a DNA-binding domain specific for a DNA sequence within human chromosome 13 that binds downstream from the genomic sequence of interest, whereby the TALEN cleaves the genomic DNA and excises the genomic sequence of interest, thereby modifying the genomic DNA of the MSC.

Another application is a method of modifying the genomic DNA of a MSC that includes introducing into the cell a first TALEN with a DNA-binding domain specific for a DNA sequence within the CLYBL safe-harbor locus that binds upstream of a genomic sequence of interest and a second TALEN with a DNA-binding domain specific for a DNA sequence within the CLYBL safe-harbor locus that binds downstream from the genomic sequence of interest, whereby the TALEN cleaves the genomic DNA and excises the genomic sequence of interest, thereby modifying the genomic DNA of the MSC.

In some embodiments, the methods include introducing a single or double-stranded donor polynucleotide with sense and/or antisense strand polynucleotide overhangs that are complementary to corresponding polynucleotide overhangs of genomic DNA into the MSC. In other embodiments, the methods include introducing a single-stranded donor polynucleotide with sense and/or antisense strand polynucleotide overhangs (regions) that are complementary to corresponding polynucleotide overhangs of genomic DNA into the MSC, wherein the overhangs are at least 15 nucleotides in length, such as 20, 30, 40, 50, 60, 70, 80, 90, 100, 200, 300, 400, 500, 600, 700, 800, 900 or 1,000 base pairs in length. The complementarity need not be 100% complementarity. For example, the complementary overhangs can be 95%, 96%, 97%, 98%, or 99% complementary to the overhangs of the cleaved DNA. In additional embodiments, the complementary overhangs are at least 98% or at least 99% homologous to the overhangs of the cleaved DNA.

One embodiment of the disclosed method of modifying the genomic DNA of a MSC involves introducing into the cell a first TALEN with a DNA-binding domain specific for a DNA sequence within an intron, such as intron 2, of the CLYBL safe-harbor locus that binds upstream of a genomic sequence of interest and a second TALEN with a DNA-binding domain specific for a DNA sequence within the intron, such as intron 2, of the CLYBL safe-harbor locus that binds downstream from the genomic sequence of interest, whereby the TALEN cleaves the genomic DNA and excises the genomic sequence of interest, thereby modifying the genomic DNA of the MSC. Another embodiment is a method of modifying the genomic DNA of a MSC that includes introducing into the MSC a first TALEN with a DNA-binding domain specific for a DNA sequence within the sequence of SEQ ID NO: 19 that binds upstream of a genomic sequence of interest and a second TALEN with a DNA-binding domain specific for a DNA sequence within the sequence of SEQ ID NO: 19 that binds downstream from the genomic sequence of interest, whereby the TALEN cleaves the genomic DNA and excises the genomic sequence of interest, thereby modifying the genomic DNA of the MSC.

Another embodiment is a method of modifying the genomic DNA of a MSC that includes introducing into the MSC a first TALEN with a DNA-binding domain specific for a DNA sequence having the sequence of SEQ ID NO: 1 that binds upstream of a genomic sequence of interest and a second TALEN with a DNA-binding domain specific for a DNA sequence having the sequence of SEQ ID NO: 3 that binds downstream from the genomic sequence of interest, whereby the TALEN cleaves the genomic DNA and excises the genomic sequence of interest, thereby modifying the genomic DNA of the MSC.

One embodiment of the method of modifying genomic DNA includes introducing into the MSC a first TALEN, having the sequence of SEQ ID NO: 7 with a DNA-binding domain specific for a DNA sequence upstream of a genomic sequence of interest and a second TALEN with a DNA-binding domain specific for a DNA sequence downstream from the genomic sequence of interest, whereby the TALEN cleaves the genomic DNA and excises the genomic sequence of interest, thereby modifying the genomic DNA of the cell. In a further embodiment, a TALEN incorporating the polypeptide of SEQ ID NO: 7 may also include a nuclease derived from fokI. In a specific embodiment, a TALEN incorporating the polypeptide of SEQ ID NO: 7 may also include a nuclease derived from fokI having the sequence of SEQ ID NO: 13.

Another embodiment of the method of modifying genomic DNA includes introducing into the MSC a first TALEN with a DNA-binding domain specific for a DNA sequence upstream of a genomic sequence of interest and a second TALEN, having the sequence of SEQ ID NO: 10, with a DNA-binding domain specific for a DNA sequence downstream from the genomic sequence of interest, whereby the TALEN cleaves the genomic DNA and excises the genomic sequence of interest, thereby modifying the genomic DNA of the MSC. In a further embodiment, a TALEN incorporating the polypeptide of SEQ ID NO: 10 may also include a nuclease derived from fokI. In a specific embodiment, a TALEN incorporating the polypeptide of SEQ ID NO: 10 may also include a nuclease derived from fokI having the sequence of SEQ ID NO: 13.

A further embodiment of the method of modifying genomic DNA includes introducing into the MSC a first TALEN, having the sequence of SEQ ID NO: 7, with a DNA-binding domain specific for a DNA sequence upstream of a genomic sequence of interest and a second TALEN, having the sequence of SEQ ID NO: 10, with a DNA-binding domain specific for a DNA sequence downstream from the genomic sequence of interest, whereby the TALEN cleaves the genomic DNA and excises the genomic sequence of interest, thereby modifying the genomic DNA of the cell. In a further embodiment, a TALEN incorporating the polypeptide of SEQ ID NO: 7 may also include a nuclease derived from fokI and a TALEN incorporating the polypeptide of SEQ ID NO: 10 may also include a nuclease derived from fokI. In a specific embodiment, a TALEN incorporating the polypeptide of SEQ ID NO: 7 may also include a nuclease derived from fokI having the sequence of SEQ ID NO: 13 and a TALEN incorporating the polypeptide of SEQ ID NO: 10 may also include a nuclease derived from fokI having the sequence of SEQ ID NO: 13.

An additional embodiment of the method of modifying genomic DNA includes introducing into the MSC a first TALEN, having the sequence of SEQ ID NO: 8, with a DNA-binding binding domain specific for a DNA sequence upstream of a genomic sequence of interest and a second TALEN with a DNA-binding domain specific for a DNA sequence downstream from the genomic sequence of interest, whereby the TALEN cleaves the genomic DNA and excises the genomic sequence of interest, thereby modifying the genomic DNA of the MSC.

One embodiment of the method of modifying genomic DNA includes introducing into the MSC a first TALEN with a DNA-binding domain specific for a DNA sequence upstream of a genomic sequence of interest and a second TALEN, having the sequence of SEQ ID NO: 11, with a DNA-binding domain specific for a DNA sequence downstream from the genomic sequence of interest, whereby the TALEN cleaves the genomic DNA and excises the genomic sequence of interest, thereby modifying the genomic DNA of the MSC.

One embodiment of the method of modifying genomic DNA includes introducing into the MSC a first TALEN, having the sequence of SEQ ID NO: 8, with a DNA-binding domain specific for a DNA sequence upstream of a genomic sequence of interest and a second TALEN, having the sequence of SEQ ID NO: 11, with a DNA-binding domain specific for a DNA sequence downstream from the genomic sequence of interest, whereby the TALEN cleaves the genomic DNA and excises the genomic sequence of interest, thereby modifying the genomic DNA of the MSC.

Methods are provided for modifying the genomic DNA of a MSC that include introducing into the cell a first TALEN with a DNA-binding domain specific for a DNA sequence within human chromosome 13 that binds to the sense strand of DNA upstream of a genomic sequence of interest and a second TALEN with a DNA-binding domain specific for a DNA sequence within human chromosome 13 that binds to the antisense strand of DNA downstream from the genomic sequence of interest. The TALEN cleave the genomic DNA and excise the genomic sequence of interest, thereby modifying the genomic DNA of the MSC.

In some embodiments, methods are provided for modifying the genomic DNA of a MSC that include introducing into the cell a first TALEN with a DNA-binding domain specific for a DNA sequence within the AAVS1 or CYBL safe-harbor locus that binds to the sense strand of DNA upstream of a genomic sequence of interest and a second TALEN with a DNA-binding domain specific for a DNA sequence within the AAVS1 or CYBL safe-harbor locus that binds to the antisense strand of DNA downstream from the genomic sequence of interest. The TALEN cleave the genomic DNA and excise the genomic sequence of interest, thereby modifying the genomic DNA of the MSC.

In additional embodiments, methods are provided for modifying the genomic DNA of a MSC that include introducing into the cell a first TALEN with a DNA-binding domain specific for a DNA sequence within an intron of the AAVS1 or CYBL safe-harbor locus that binds to the sense strand of DNA upstream of a genomic sequence of interest and a second TALEN with a DNA-binding domain specific for a DNA sequence within the AAVS1 or CYBL safe-harbor locus that binds to the antisense strand of DNA downstream from the genomic sequence of interest. The TALEN cleave the genomic DNA and excise the genomic sequence of interest, thereby modifying the genomic DNA of the MSC.

In further embodiments, methods are provided for modifying the genomic DNA of a MSC that include introducing into the MSC a first TALEN with a DNA-binding domain specific for a DNA sequence within the sequence of SEQ ID NO: 19 that binds to the sense strand of DNA upstream of a genomic sequence of interest and a second TALEN with a DNA-binding domain specific for a DNA sequence within the sequence of SEQ ID NO: 19 that binds to the antisense strand of DNA downstream from the genomic sequence of interest. The TALEN cleave the genomic DNA and excise the genomic sequence of interest, thereby modifying the genomic DNA of the MSC.

In further embodiments, method are provided for modifying the genomic DNA of a MSC that include introducing into the cell a first TALEN with a DNA-binding domain specific for a DNA sequence having the sequence of SEQ ID NO: 1 that binds to the sense strand of DNA upstream of a genomic sequence of interest and a second TALEN with a DNA-binding domain specific for a DNA sequence having the sequence of SEQ ID NO: 3 that binds to the antisense strand of DNA downstream from the genomic sequence of interest. The TALEN cleave the genomic DNA and excise the genomic sequence of interest, thereby modifying the genomic DNA of the MSC.

In some embodiments, methods are provided for modifying genomic DNA that include introducing into the MSC a first TALEN, having the sequence of SEQ ID NO: 7, with a DNA-binding domain specific for a DNA sequence on the sense strand of DNA upstream of a genomic sequence of interest and a second TALEN with a DNA-binding domain specific for a DNA sequence on the antisense strand of DNA downstream from the genomic sequence of interest. The TALEN cleave the genomic DNA and excise the genomic sequence of interest, thereby modifying the genomic DNA of the cell. In a further embodiment, a TALEN incorporating the polypeptide of SEQ ID NO: 7 may also include a nuclease derived from fokI. In a specific embodiment, a TALEN incorporating the polypeptide of SEQ ID NO: 7 may also include a nuclease derived from fokI having the sequence of SEQ ID NO: 13.

One embodiment of the method of modifying genomic DNA includes introducing into the MSC a first TALEN with a DNA-binding domain specific for a DNA sequence on the sense strand of DNA upstream of a genomic sequence of interest and a second TALEN, having the sequence of SEQ ID NO: 10, with a DNA-binding domain specific for a DNA sequence on the antisense strand of DNA downstream from the genomic sequence of interest. The TALEN cleave the genomic DNA and excise the genomic sequence of interest, thereby modifying the genomic DNA of the MSC. In a further embodiment, a TALEN incorporating the polypeptide of SEQ ID NO: 10 may also include a nuclease derived from fokI. In a specific embodiment, a TALEN incorporating the polypeptide of SEQ ID NO: 10 may also include a nuclease derived from fokI having the sequence of SEQ ID NO: 13.

One embodiment of the method of modifying genomic DNA includes introducing into the MSC a first TALEN, having the sequence of SEQ ID NO: 7, with a DNA-binding domain specific for a DNA sequence on the sense strand of DNA upstream of a genomic sequence of interest and a second TALEN, having the sequence of SEQ ID NO: 10, with a DNA-binding domain specific for a DNA sequence on the antisense strand of DNA downstream from the genomic sequence of interest. The TALEN cleave the genomic DNA and excise the genomic sequence of interest, thereby modifying the genomic DNA of the cell. In a further embodiment, a TALEN incorporating the polypeptide of SEQ ID NO: 7 may also include a nuclease derived from fokI and a TALEN incorporating the polypeptide of SEQ ID NO: 10 may also include a nuclease derived from fokI. In a specific embodiment, a TALEN incorporating the polypeptide of SEQ ID NO: 7, may also include a nuclease derived from fokI having the sequence of SEQ ID NO: 13 and a TALEN incorporating the polypeptide of SEQ ID NO: 10 may also include a nuclease derived from fokI having the sequence of SEQ ID NO: 13.

One embodiment of the method of modifying genomic DNA includes introducing into the MSC a first TALEN, having the sequence of SEQ ID NO: 8, with a DNA-binding domain specific for a DNA sequence on the sense strand of DNA upstream of a genomic sequence of interest and a second TALEN with a DNA-binding domain specific for a DNA sequence on the antisense strand of DNA downstream from the genomic sequence of interest. The TALEN cleave the genomic DNA and excise the genomic sequence of interest, thereby modifying the genomic DNA of the cell.

Another embodiment of the method of modifying genomic DNA includes introducing into the MSC a first TALEN with a DNA-binding domain specific for a DNA sequence on the sense strand of DNA upstream of a genomic sequence of interest and a second TALEN, having the sequence of SEQ ID NO: 11, with a DNA-binding domain specific for a DNA sequence on the antisense strand of DNA downstream from the genomic sequence of interest. The TALEN cleave the genomic DNA and excise the genomic sequence of interest, thereby modifying the genomic DNA of the cell.

Yet another embodiment of the method of modifying genomic DNA includes introducing into the MSC a first TALEN, having the sequence of SEQ ID NO: 8, with a DNA-binding domain specific for a DNA sequence on the sense strand of DNA upstream of a genomic sequence of interest and a second TALEN, having the sequence of SEQ ID NO: 11, with a DNA-binding domain specific for a DNA sequence on the antisense strand of DNA downstream from the genomic sequence of interest. The TALEN cleaves the genomic DNA and excises the genomic sequence of interest, thereby modifying the genomic DNA of the MSC.

In accordance with the methods of modifying genomic DNA described in this section it should be understood that broadly applicable further aspects of these methods may be carried out as needed or desired. In one embodiment the described methods can be carried out to cause polynucleotide excision in both copies of the same chromosome.

Provided herein are methods of using the described polynucleotide-binding polypeptides, the recombinant DNA-binding polypeptides, zinc-finger or TALE domains, nuclease proteins or polypeptides, fusion proteins produced from the fusion of polynucleotide-binding polypeptides and nuclease proteins or polypeptides, and TALENs for inserting a polynucleotide into the genome of a MSC. In some embodiments, the method is carried out by introducing into a MSC a first polypeptide with a DNA-binding domain specific for a DNA sequence upstream of a genomic sequence of interest, a second polypeptide with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced polypeptides at a genomic insertion site. The complementary overhangs facilitate insertion of the donor polynucleotide to the cleaved genomic DNA, providing for the introduction of the donor polynucleotide into the genome of the MSC.

One embodiment of the disclosed method is carried out by introducing into a MSC a first polypeptide with a DNA-binding domain, including the sequence of SEQ ID NO: 7, specific for a DNA sequence upstream of a genomic sequence of interest, a second polypeptide with a DNA-binding domain, including the sequence of SEQ ID NO: 10, specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced polypeptides at a genomic insertion site. The complementary overhangs facilitate insertion of the donor polynucleotide to the cleaved genomic DNA, providing for the introduction of the donor polynucleotide into the genome of the MSC.

In some embodiments, the method includes introducing into a MSC a first polypeptide with a DNA-binding domain specific for a DNA sequence within the sequence of SEQ ID NO: 19 upstream of a genomic sequence of interest, a second polypeptide with a DNA-binding domain specific for a DNA sequence within the sequence of SEQ ID NO: 19 downstream from the genomic sequence of interest, and a single or double-stranded donor polynucleotide with sense and/or antisense strand polynucleotide overhangs that are complementary to corresponding polynucleotide overhangs of genomic DNA cleaved by the introduced polypeptides at a genomic insertion site. The complementary overhangs facilitate insertion of the donor polynucleotide to the cleaved genomic DNA, providing for the introduction of the donor polynucleotide into the genome of the MSC.

In some embodiments, the methods include introducing into a MSC a first TALEN with a DNA-binding domain specific for a DNA sequence upstream of a genomic sequence of interest, a second TALEN with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

Methods are also provided for using the polynucleotide-binding polypeptides, the recombinant DNA-binding polypeptides, zinc-finger or TALE domains, nuclease proteins or polypeptides, fusion proteins produced from the fusion of polynucleotide-binding polypeptides and nuclease proteins or polypeptides, and TALENs. One application is a method of inserting a polynucleotide into the genome of a MSC by introducing into the MSC a first TALEN with a DNA-binding domain specific for a DNA sequence upstream of a genomic sequence of interest and a second TALEN with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

Methods are also provided for inserting a polynucleotide into the genome of a MSC by introducing into the MSC a first TALEN with a DNA-binding domain specific for a DNA sequence within human chromosome 13 that binds upstream of a genomic sequence of interest and a second TALEN with a DNA-binding domain specific for a DNA sequence within human chromosome 13 that binds downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

In some embodiments, methods are provided for inserting a polynucleotide into the genome of a MSC by introducing into the MSC a first TALEN with a DNA-binding domain specific for a DNA sequence within the AAVS1 safe-harbor locus that binds upstream of a genomic sequence of interest and a second TALEN with a DNA-binding domain specific for a DNA sequence within the AAVS1 safe-harbor locus that binds downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the described method of inserting a polynucleotide into the genome of a MSC involves introducing into the MSC a first TALEN with a DNA-binding domain specific for a DNA sequence within an intron (such as intron 2) of the AAVS1 or CYBL safe-harbor locus that binds upstream of a genomic sequence of interest and a second TALEN with a DNA-binding domain specific for a DNA sequence within the AAVS1 or CYBL safe-harbor locus that binds downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

Another application is a method of inserting a polynucleotide into the genome of a MSC by introducing into the MSC a first TALEN with a DNA-binding domain specific for a DNA sequence within the sequence of SEQ ID NO: 19 that binds upstream of a genomic sequence of interest and a second TALEN with a DNA-binding domain specific for a DNA sequence within the sequence of SEQ ID NO: 19 that binds downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

In some embodiments, methods are provided for inserting a polynucleotide into the genome of a MSC by introducing into the MSC a first TALEN with a DNA-binding domain specific for a DNA sequence having the sequence of SEQ ID NO: 1 that binds upstream of a genomic sequence of interest and a second TALEN with a DNA-binding domain specific for a DNA sequence having the sequence of SEQ ID NO: 3 that binds downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the method of inserting a polynucleotide into the genome of a MSC includes introducing into the MSC a first TALEN, having the sequence of SEQ ID NO: 7, with a DNA-binding domain specific for a DNA sequence upstream of a genomic sequence of interest and a second TALEN with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI. In a specific embodiment, a TALEN incorporating the polypeptide of SEQ ID NO: 7 may also include a nuclease derived from fokI having the sequence of SEQ ID NO: 13.

One embodiment of the method of inserting a polynucleotide into the genome of a MSC includes introducing into the MSC a first TALEN with a DNA-binding domain specific for a DNA sequence upstream of a genomic sequence of interest and a second TALEN, having the sequence of SEQ ID NO: 10, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 10 may also include a nuclease derived from fokI. In a specific embodiment, a TALEN incorporating the polypeptide of SEQ ID NO: 10 may also include a nuclease derived from fokI having the sequence of SEQ ID NO: 13.

One embodiment of the method of inserting a polynucleotide into the genome of a MSC includes introducing into the MSC a first TALEN, having the sequence of SEQ ID NO: 7, with a DNA-binding domain specific for a DNA sequence upstream of a genomic sequence of interest and a second TALEN, having the sequence of SEQ ID NO: 10, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI and a TALEN incorporating the polypeptide of SEQ ID NO: 10 may also include a nuclease derived from fokI. In a specific embodiment, a TALEN incorporating the polypeptide of SEQ ID NO: 7 may also include a nuclease derived from fokI having the sequence of SEQ ID NO: 13 and a TALEN incorporating the polypeptide of SEQ ID NO: 10 may also include a nuclease derived from fokI having the sequence of SEQ ID NO: 13.

One embodiment of the method of inserting a polynucleotide into the genome of a MSC includes introducing into the MSC a first TALEN, having the sequence of SEQ ID NO: 8, with a DNA-binding domain specific for a DNA sequence upstream of a genomic sequence of interest and a second TALEN with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the method of inserting a polynucleotide into the genome of a MSC includes introducing into the MSC a first TALEN with a DNA-binding domain specific for a DNA sequence upstream of a genomic sequence of interest and a second TALEN, having the sequence of SEQ ID NO: 11, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the method of inserting a polynucleotide into the genome of a MSC includes introducing into the MSC a first TALEN, having the sequence of SEQ ID NO: 8, with a DNA-binding domain specific for a DNA sequence upstream of a genomic sequence of interest and a second TALEN, having the sequence of SEQ ID NO: 11, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

In some embodiments, a method of inserting a polynucleotide into the genome of a MSC includes introducing into the MSC a first TALEN with a DNA-binding domain specific for a DNA sequence within human chromosome 13 that binds to the sense strand of DNA upstream of a genomic sequence of interest and a second TALEN with a DNA-binding domain specific for a DNA sequence within human chromosome 13 that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

In additional embodiments, methods are provided for inserting a polynucleotide into the genome of a MSC that include introducing into the MSC a first TALEN with a DNA-binding domain specific for a DNA sequence within the AAVS1 or CYBL safe-harbor locus that binds to the sense strand of DNA upstream of a genomic sequence of interest and a second TALEN with a DNA-binding domain specific for a DNA sequence within the AAVS1 or CYBL safe-harbor locus that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the described method of inserting a polynucleotide into the genome of a MSC involves introducing into the MSC a first TALEN with a DNA-binding domain specific for a DNA sequence within the AAVS1 or CYBL safe-harbor locus that binds to the sense strand of DNA upstream of a genomic sequence of interest and a second TALEN with a DNA-binding domain specific for a DNA sequence within intron 1 of the AAVS1 safe harbor locus or intron 2 of the CLYBL safe-harbor locus that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

In additional embodiments, methods are provided for inserting a polynucleotide into the genome of a MSC that include introducing into the MSC a first TALEN with a DNA-binding domain specific for a DNA sequence within the sequence of SEQ ID NO: 19 that binds to the sense strand of DNA upstream of a genomic sequence of interest and a second TALEN with a DNA-binding domain specific for a DNA sequence within the sequence of SEQ ID NO: 19 that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

In further embodiments, methods are provided for inserting a polynucleotide into the genome of a MSC that include introducing into the MSC a first TALEN with a DNA-binding domain specific for a DNA sequence having the sequence of SEQ ID NO: 1 that binds to the sense strand of DNA upstream of a genomic sequence of interest and a second TALEN with a DNA-binding domain specific for a DNA sequence having the sequence of SEQ ID NO: 3 that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the method of inserting a polynucleotide into the genome of a MSC includes introducing into the MSC a first TALEN, having the sequence of SEQ ID NO: 7, with a DNA-binding domain specific for a DNA sequence on the sense strand of DNA upstream of a genomic sequence of interest and a second TALEN with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI. In a specific embodiment, a TALEN incorporating the polypeptide of SEQ ID NO: 7 may also include a nuclease derived from fokI having the sequence of SEQ ID NO: 13.

One embodiment of the method of inserting a polynucleotide into the genome of a MSC includes introducing into the MSC a first TALEN with a DNA-binding domain specific for a DNA sequence on the sense strand of DNA upstream of a genomic sequence of interest and a second TALEN, having the sequence of SEQ ID NO: 10, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 10 may also include a nuclease derived from fokI. In a specific embodiment, a TALEN incorporating the polypeptide of SEQ ID NO: 10 may also include a nuclease derived from fokI having the sequence of SEQ ID NO: 13.

One embodiment of the method of inserting a polynucleotide into the genome of a MSC includes introducing into the MSC a first TALEN, having the sequence of SEQ ID NO: 7, with a DNA-binding domain specific for a DNA sequence on the sense strand of DNA upstream of a genomic sequence of interest and a second TALEN, having the sequence of SEQ ID NO: 10, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI and a TALEN incorporating the polypeptide of SEQ ID NO: 10 may also include a nuclease derived from fokI. In a specific embodiment, a TALEN incorporating the polypeptide of SEQ ID NO: 7 may also include a nuclease derived from fokI having the sequence of SEQ ID NO: 13 and a TALEN incorporating the polypeptide of SEQ ID NO: 10 may also include a nuclease derived from fokI having the sequence of SEQ ID NO: 13.

One embodiment of the method of inserting a polynucleotide into the genome of a MSC includes introducing into the MSC a first TALEN, having the sequence of SEQ ID NO: 8, with a DNA-binding domain specific for a DNA sequence on the sense strand of DNA upstream of a genomic sequence of interest and a second TALEN with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the method of inserting a polynucleotide into the genome of a MSC includes introducing into the MSC a first TALEN with a DNA-binding domain specific for a DNA sequence on the sense strand of DNA upstream of a genomic sequence of interest and a second TALEN, having the sequence of SEQ ID NO: 11, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

One embodiment of the method of inserting a polynucleotide into the genome of a MSC includes introducing into the MSC a first TALEN, having the sequence of SEQ ID NO: 8, with a DNA-binding domain specific for a DNA sequence on the sense strand of DNA upstream of a genomic sequence of interest and a second TALEN, having the sequence of SEQ ID NO: 11, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC.

In accordance with the methods of inserting a polynucleotide into the genome of a MSC described in this section it should be understood that broadly applicable further aspects of these methods may be carried out as needed or desired. In one embodiment the described methods can be carried out to cause polynucleotide excision in both copies of the same chromosome.

In some embodiments the step of introducing a first polypeptide or TALEN into a MSC involves transfecting the MSC with a polynucleotide encoding the polypeptide or TALEN. In some embodiments the step of introducing a second polypeptide or TALEN into a MSC involves transfecting the MSC with a polynucleotide encoding the polypeptide or TALEN. In some embodiments a single vector may be used to transfect a MSC with polynucleotides that encode an upstream TALEN and the nucleic acid encoding the downstream TALEN.

### Differentiation of MSCs

Methods are provided for inducing MSCs to differentiate to selected mature cells and/or tissues including, but not limited, adipocytes, cartilage, bone, tendons, muscle, and skin as well as myocytes, neurons and glia.

In some embodiments, the method includes expressing an agent for inducing the proliferation and/or differentiation of the MSC into the selected mature cells and/or tissues. The agent can be a trophic agent or a growth factor. The agent or growth factor can be encoded by the polynucleotide of interest.

In specific non-limiting examples, the agent is nerve growth factor, nerve growth factor, insulin, fibroblast growth factor, glial derived neurotropic factor, a Notch ligand, Delta, brain derived neurotrophic factor, glial derived neurotrophic factor, bone morphogenic protein-2 or 4 (BMP-2/4), cilliarly neurotrophic factor (CNTF), heregulin-1 beta, platelet derived growth factor (PDGF)-1 or PDGF-B. In other embodiments, the donor polynucleotide also encodes a selectable marker and/or a detectable label. Suitable detectable labels include, but are not limited to, enzymes such as horse radish peroxidase and alkaline phosphatase, and fluorescent proteins, such as green fluorescent protein.

The donor polynucleotide can include a promoter operably linked to a heterologous nucleic acid, such as a nucleic acid encoding an agent of interest and/or a selectable marker and/or a detectable marker. The promoter can be constitutive or inducible. The promoter can be a lineage specific promoter, such as a promoter suitable for expression in adipocytes, cartilage, bone, tendons, muscle, and skin as well as myocytes, neurons and glia. In specific non-limiting examples, the promoter is a doublecourtin or a GFAP promoter.

Methods for introducing DNA into MSCs include chemical and physical methods. Chemical methods include liposome-based gene transfer or lipofection, calcium phosphate-mediated gene transfer, DEAE-dextran transfection techniques, and polyethyleneimine (PEI)-mediated delivery. Physical methods include ballistic gene transfer, microinjection, and nucleofection (Amaxa biosystem, 2004). In some embodiments, nucleofection can be used to introduce the polynucleotides disclosed herein into MSCs. In a specific non-limiting example, the nucleofection involves the use of a nucleofectin D apparatus. In some embodiments, the nucleofection provides a transfection efficiency of at least about 60%, at least about 65%, at least about 70%, at least about 75%, at least about 80%, at least about 85%, at least about 90%, or at least about 95%. In specific non-limiting example, the transfection efficiency is at least about 80%, at least about 85%, at least about 90%, at least about 95%, at least about 96%, about 97%, about 98% or about 99%.

The method can include contacting the mesenchymal stem cell with the upstream TALEN, the downstream TALEN, and the polynucleotide of interest at a ratio of about 1:1:1. In additional embodiments, a ratio of about 1:2:1 or 2:1:1 or 1:1:2 is utilized. In other embodiments, 1:3:1 or 3:1:1 or 1:1:3 is utilized. In yet other embodiments a ratio of 1:4:1 or 4:1:1 or 1:4:1 I is utilized. In further embodiments a ratio of 1:5:1 or 5:1:1 or 1:1:5 is utilized.

In some embodiments, these methods include introducing into the MSC (a) an upstream transcription activator-like effector nuclease (TALEN) comprising an upstream DNA-binding domain linked to a DNA cleavage domain, wherein the upstream DNA binding domain specifically binds to the safe-harbor locus at a site upstream of a genomic insertion site in the genome of the MSC, (b) a downstream transcription activator-like effector nuclease (TALEN) comprising a downstream DNA-binding domain linked to a DNA cleavage domain, wherein the downstream DNA binding domain specifically binds to the safe-harbor locus at a site downstream of the genomic insertion site in the genome of the MSC, and (c) a single or double-stranded donor polynucleotide comprising sense and/or antisense strand polynucleotide overhangs that are complementary to corresponding polynucleotide overhangs of cleaved the genomic DNA when cleaved at the genomic insertion site. The complementary overhangs facilitate homologous recombination of the donor polynucleotide with the cleaved genomic DNA, thereby introducing the donor polynucleotide into the genome of the MSC. In some embodiments, the upstream TALEN binds to the sense strand of a genomic DNA locus flanking the insertion site and the downstream TALEN binds to the antisense strand of a genomic DNA locus flanking the insertion site. In additional embodiments, the donor polynucleotide encodes one or more agents sufficient to differentiate the MSC selected mature cells and/or tissues including, but not limited, adipocytes, cartilage, bone, tendons, muscle, and skin as well as myocytes, neurons and glia.

The complementary overhangs facilitate homologous recombination of the donor polynucleotide with the cleaved genomic DNA, to allow introduction of the polynucleotide into the genome of the MSC. These methods provide introduction of the donor polynucleotide into the genomic insertion site into the safe harbor locus in the genome of the MSC. In some embodiments, the upstream TALEN binds to the sense strand of a genomic DNA locus flanking the insertion site and the downstream TALEN binds to the antisense strand of a genomic DNA locus flanking the insertion site. In some embodiments, the donor polynucleotide encodes one or more agents sufficient to differentiate the MSC into the selected mature cells and/or tissues including, but not limited, adipocytes, cartilage, bone, tendons, muscle, and skin as well as myocytes, neurons and glia.

In some embodiments the upstream TALEN comprises SEQ ID NO:8. In additional embodiments, the downstream TALEN comprises SEQ ID NO:11. In further embodiments, the DNA cleavage domain comprises a FokI nuclease domain, such as, but not limited to, SEQ ID NO: 13. In some embodiments, the genomic sense strand locus bound by the upstream TALEN comprises SEQ ID NO: 1. In yet other embodiments, the genomic antisense strand locus bound by the downstream TALEN comprises SEQ ID NO: 3. In further embodiments, the donor polynucleotide is inserted into both copies of the same chromosome. In some embodiments, the polynucleotide is inserted into the two copies of the same chromosome. In some embodiments, the donor polynucleotide encodes one or more factor sufficient to differentiate the MSCs into selected mature cells and/or tissues including, but not limited, adipocytes, cartilage, bone, tendons, muscle, and skin as well as myocytes, neurons and glia.

A further embodiment of the method of differentiating a MSC includes introducing into the MSC a first TALEN, having the sequence of SEQ ID NO: 7, with a DNA-binding domain specific for a DNA sequence upstream of a genomic sequence of interest and a second TALEN, having the sequence of SEQ ID NO: 10, with a DNA-binding domain specific for a DNA sequence downstream from the genomic sequence of interest, whereby the TALEN cleaves the genomic DNA and excises the genomic sequence of interest, thereby modifying the genomic DNA of the MSC. In a further embodiment, a TALEN incorporating the polypeptide of SEQ ID NO: 7 may also include a nuclease derived from fokI and a TALEN incorporating the polypeptide of SEQ ID NO: 10 may also include a nuclease derived from fokI. In a specific embodiment, a TALEN incorporating the polypeptide of SEQ ID NO: 7 may also include a nuclease derived from fokI having the sequence of SEQ ID NO: 13 and a TALEN incorporating the polypeptide of SEQ ID NO: 10 may also include a nuclease derived from fokI having the sequence of SEQ ID NO: 13. In some embodiments, the donor polynucleotide encodes one or more agents sufficient to differentiate the MSCs into selected mature cells and/or tissues including, but not limited, adipocytes, cartilage, bone, tendons, muscle, and skin as well as myocytes, neurons and glia.

In some embodiments, methods for differentiating an MSC include introducing into the MSC a first TALEN with a DNA-binding domain specific for a DNA sequence having the sequence of SEQ ID NO: 1 that binds upstream of a genomic sequence of interest and a second TALEN with a DNA-binding domain specific for a DNA sequence having the sequence of SEQ ID NO: 3 that binds downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSCs into selected mature cells and/or tissues including, but not limited, adipocytes, cartilage, bone, tendons, muscle, and skin as well as myocytes, neurons and glia.

One embodiment of the method of differentiating a MSC includes introducing into the MSC a first TALEN, having the sequence of SEQ ID NO: 7, with a DNA-binding domain specific for a DNA sequence upstream of a genomic sequence of interest and a second TALEN with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI. In a specific embodiment, a TALEN incorporating the polypeptide of SEQ ID NO: 7 may also include a nuclease derived from fokI having the sequence of SEQ ID NO: 13. The donor polynucleotide encodes one or more agents, wherein expression of the one or more agents is sufficient to differentiate the MSCs into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN with a DNA-binding domain specific for a DNA sequence upstream of a genomic sequence of interest and a second TALEN, having the sequence of SEQ ID NO: 10, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 10 may also include a nuclease derived from fokI. In a specific embodiment, a TALEN incorporating the polypeptide of SEQ ID NO: 10 may also include a nuclease derived from fokI having the sequence of SEQ ID NO: 13. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN, having the sequence of SEQ ID NO: 7, with a DNA-binding domain specific for a DNA sequence upstream of a genomic sequence of interest and a second TALEN, having the sequence of SEQ ID NO: 10, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI and a TALEN incorporating the polypeptide of SEQ ID NO: 10 may also include a nuclease derived from fokI. In a specific embodiment, a TALEN incorporating the polypeptide of SEQ ID NO: 7 may also include a nuclease derived from fokI having the sequence of SEQ ID NO: 13 and a TALEN incorporating the polypeptide of SEQ ID NO: 10 may also include a nuclease derived from fokI having the sequence of SEQ ID NO: 13. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN, having the sequence of SEQ ID NO: 8, with a DNA-binding domain specific for a DNA sequence upstream of a genomic sequence of interest and a second TALEN with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

One embodiment of the method of the method for differentiating a MSC includes introducing into the MSC a first TALEN with a DNA-binding domain specific for a DNA sequence upstream of a genomic sequence of interest and a second TALEN, having the sequence of SEQ ID NO: 11, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to 30, differentiate the MSC into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN, having the sequence of SEQ ID NO: 8, with a DNA-binding domain specific for a DNA sequence upstream of a genomic sequence of interest and a second TALEN, having the sequence of SEQ ID NO: 11, with a DNA-binding domain specific for a DNA sequence downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSCs into selected mature cells and/or tissues including, but not limited, adipocytes, cartilage, bone, tendons, muscle, and skin as well as myocytes, neurons and glia.

In some embodiments, methods are provided for differentiating a MSC that include introducing into the MSC a first TALEN with a DNA-binding domain specific for a DNA sequence within human chromosome 13 that binds to the sense strand of DNA upstream of a genomic sequence of interest and a second TALEN with a DNA-binding domain specific for a DNA sequence within human chromosome 13 that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

In some embodiments, methods are provided for differentiating a MSC that include introducing into the MSC a first TALEN with a DNA-binding domain specific for a DNA sequence within the AAVS1 or CYBL safe-harbor locus that binds to the sense strand of DNA upstream of a genomic sequence of interest and a second TALEN with a DNA-binding domain specific for a DNA sequence within the AAVS1 or CYBL safe-harbor locus that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into the selected mature cells and/or tissues including, but not limited, adipocytes, cartilage, bone, tendons, muscle, and skin as well as myocytes, neurons and glia.

One embodiment for differentiating an MSC includes introducing into the MSC a first TALEN with a DNA-binding domain specific for a DNA sequence within intron 1 of the AAVS1 safe-harbor locus or intron 2 of the CYBL safe harbor locus that binds to the sense strand of DNA upstream of a genomic sequence of interest and a second TALEN with a DNA-binding domain specific for a DNA sequence within intron 1 of the AAVS1 safe-harbor locus or intron 2 of the CYBL safe-harbor locus that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

Another application is a method for differentiating a MSC by introducing into the MSC a first TALEN with a DNA-binding domain specific for a DNA sequence within the sequence of SEQ ID NO: 19 that binds to the sense strand of DNA upstream of a genomic sequence of interest and a second TALEN with a DNA-binding domain specific for a DNA sequence within the sequence of SEQ ID NO: 19 that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

Another application is a method for differentiating a MSC that includes introducing into the MSC a first TALEN with a DNA-binding domain specific for a DNA sequence having the sequence of SEQ ID NO: 1 that binds to the sense strand of DNA upstream of a genomic sequence of interest and a second TALEN with a DNA-binding domain specific for a DNA sequence having the sequence of SEQ ID NO: 3 that binds to the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons, and glia.

One embodiment of the method for differentiating a MSC includes introducing into the MSC a first TALEN, having the sequence of SEQ ID NO: 7, with a DNA-binding domain specific for a DNA sequence on the sense strand of DNA upstream of a genomic sequence of interest and a second TALEN with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI. In a specific embodiment, a TALEN incorporating the polypeptide of SEQ ID NO: 7 may also include a nuclease derived from fokI having the sequence of SEQ ID NO: 13. The donor polynucleotide encodes one or more agents, wherein expression of the one or more agents is sufficient to differentiate the MSCs into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN with a DNA-binding domain specific for a DNA sequence on the sense strand of DNA upstream of a genomic sequence of interest and a second TALEN, having the sequence of SEQ ID NO: 10, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 10 may also include a nuclease derived from fokI. In a specific embodiment, a TALEN incorporating the polypeptide of SEQ ID NO: 10 may also include a nuclease derived from fokI having the sequence of SEQ ID NO: 13. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN, having the sequence of SEQ ID NO: 7, with a DNA-binding domain specific for a DNA sequence on the sense strand of DNA upstream of a genomic sequence of interest and a second TALEN, having the sequence of SEQ ID NO: 10, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. In a further embodiment, a TALEN incorporating the polypeptide of SEQ ID NO: 7 may also include a nuclease derived from fokI and a TALEN incorporating the polypeptide of SEQ ID NO: 10 may also include a nuclease derived from fokI. In a specific embodiment, a TALEN incorporating the polypeptide of SEQ ID NO: 7 may also include a nuclease derived from fokI having the sequence of SEQ ID NO: 13 and a TALEN incorporating the polypeptide of SEQ ID NO: 10 may also include a nuclease derived from fokI having the sequence of SEQ ID NO: 13. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN, having the sequence of SEQ ID NO: 8, with a DNA-binding domain specific for a DNA sequence on the sense strand of DNA upstream of a genomic sequence of interest and a second TALEN with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN with a DNA-binding domain specific for a DNA sequence on the sense strand of DNA upstream of a genomic sequence of interest and a second TALEN, having the sequence of SEQ ID NO: 11, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

One embodiment of the method for differentiating a MSC includes introducing into the MSC a first TALEN, having the sequence of SEQ ID NO: 8, with a DNA-binding domain specific for a DNA sequence on the sense strand of DNA upstream of a genomic sequence of interest and a second TALEN, having the sequence of SEQ ID NO: 11, with a DNA-binding domain specific for a DNA sequence on the antisense strand of DNA downstream from the genomic sequence of interest, and a single or double-stranded donor polynucleotide with sense and/or antisense strand polynucleotide overhangs that are complementary to corresponding polynucleotide overhangs of genomic DNA cleaved by the introduced TALENs at a genomic insertion site. The complementary overhangs facilitate homologous recombination of the donor polynucleotide with the cleaved genomic DNA, providing for the introduction of the donor polynucleotide into the genome of the MSC. The donor polynucleotide encodes one or more agents, wherein expression of the one or more agents is sufficient to differentiate the MSC into selected mature cells and/or tissues including, but not limited, adipocytes, cartilage, bone, tendons, muscle, and skin as well as myocytes, neurons and glia.

In accordance with the methods of inserting a polynucleotide into the genome of a MSC described in this section it should be understood that broadly applicable further aspects of these methods may be carried out as needed or desired. In one embodiment the described methods can be carried out to cause polynucleotide excision in both copies of the same chromosome. In one embodiment the described methods can be carried out using nuclofection of a polynucleotide or vectors encoding the polypeptides or TALENs used with these methods. In some embodiments the step of introducing a first polypeptide or TALEN into a MSC involves transfecting the MSC with a polynucleotide encoding the polypeptide or TALEN. In some embodiments the step of introducing a second polypeptide or TALEN into a MSC involves transfecting the MSC with a polynucleotide encoding the polypeptide or TALEN. In some embodiments a single vector may be used to transfect a MSC with polynucleotides that encode an upstream TALEN and the nucleic acid encoding the downstream TALEN.

In accordance with the methods of inserting a polynucleotide into the genome of a MSC described in this section it should be understood that broadly applicable further aspects of these methods may be carried out as needed or desired. In one embodiment the described methods can be carried out to cause polynucleotide excision in both copies of the same chromosome.

### Methods of Treatment

The methods disclosed herein modify the genome of a MSC and/or differentiate the MSC. The MSCs, and cells differentiated from these MSCs, can be used for treating a subject.

In some embodiments, the disclosed methods can be employed to produce MSCs and/or selected differentiated mature cells produced from these MSCs in order to deliver the cells, or molecules expressed by these cells, to a subject in need thereof.

In one nonlimiting embodiment, the subject may be suffering from a disease or disorder such as, but not limited to, an inflammatory or immune disease or disorder, a neurological disease or disorder, cancer or a cardiovascular disease or disorder.

In one nonlimiting embodiment, the subject may be suffering from a disease or disorder relating to absence of a protein such as an enzyme in, for example, lysosomal storage disorders, a growth factor useful, for example, in enhancing bone regrowth and/or accelerating ulcer repair or limb ischemia, or a cytokine useful in alleviating pain relating to an immune disorder such as rheumatoid arthritis.

In yet another nonlimiting embodiment, the genome of the MSC may be modified to produce an antibody, useful in treating a disease or disorder wherein antibody treatment is warranted.

Examples of disease or disorders expected to be treatable with MSCs modified in accordance with the present invention include, but are in no way limited to, cancer, autoimmune disease including, but in no way limited to, rheumatoid arthritis, multiple sclerosis, Crohn's disease, lupus and psoriasis, high cholesterol, organ transplant to prevent rejection, cardiovascular disease, stroke, Alzheimer's disease, bone diseases, sepsis, infectious diseases, viral infections, blood disorders, osteoporosis and asthma.

After the mesenchymal stem cells are formed and/or differentiated according to the methods disclosed above, the cells are suspended in a physiologically compatible carrier. The carrier can be any carrier compatible with the other ingredients of the formulation and not deleterious to the recipient thereof. Those of skill in the art are familiar with physiologically compatible carriers. Examples of suitable carriers include cell culture medium (e.g., Eagle's minimal essential media), phosphate buffered saline, and Hank's balanced salt solution+/−glucose (HBSS). In one embodiment, supporting cells, such as glia or astrocytes, can be added. These cells can be from the same species as the mesenchymal stem cells, or from a different species. Thus, in one nonlimiting embodiment, mesenchymal stem cells are differentiated to neuronal cells, and administered to the subject in conjunction with human glia or astrocytes. In some embodiments, the coadministered cells can be non-human.

The volume of cell suspension administered to a subject will vary depending on the site of implantation, treatment goal and amount of cells in solution. Typically the amount of cells administered to a subject will be a therapeutically effective amount. For example, where the treatment is for a neurodegenerative condition such as Parkinson's disease, transplantation of a therapeutically effective amount of cells will typically produce a reduction in the amount and/or severity of the symptoms associated with that disorder, e.g., rigidity, akinesia and gait disorder.

### Screening Methods

It should be noted that cells produced by the methods disclosed herein can also be used in to screen pharmaceutical agents to select for agents that affect specific human cell types, such as agents that affect mesenchymal stem cells or derivatives thereof.

In some embodiments, methods are provided for assessing the physiological effect of a polypeptide on a MSC. The methods include introducing into a polynucleotide into the MSC using any of the methods disclosed above, and assessing a parameter of the mesenchymal stem cell, thereby determining the physiological effect of the polypeptide on the MSC.

In some embodiments, methods are provided for assessing the physiological effect of a polypeptide on a MSC. The methods include excising a polynucleotide from the MSC using any of the methods disclosed above, and assessing a parameter of the MSC, thereby determining the physiological effect of the polypeptide on the MSC.

A method is provided herein for selecting an agent that affects the differentiation of human MSCs. In one embodiment, the agent affects the differentiation of human MSCs into a differentiated cell fate.

The test compound can be any compound of interest, including chemical compounds, small molecules, polypeptides or other biological agents (for example antibodies or cytokines). In several examples, a panel of potential agents is screened, such as a panel of cytokines or growth factors is screened.

Methods for preparing a combinatorial library of molecules that can be tested for a desired activity are well known in the art and include, for example, methods of making a phage display library of peptides, which can be constrained peptides (see, for example, U.S. Pat. No. 5,622,699; U.S. Pat. No. 5,206,347; Scott and Smith, Science 249:386-390, 1992; Markland et al., Gene 109:13-19, 1991), a peptide library (U.S. Pat. No. 5,264,563); a peptidomimetic library (Blondelle et al., Trends Anal Chem. 14:83-92, 1995); a nucleic acid library (O'Connell et al., Proc. Natl Acad. Sci., USA 93:5883-5887, 1996; Tuerk and Gold, Science 249:505-510, 1990; Gold et al., Ann. Rev. Biochem. 64:763-797, 1995); an oligosaccharide library (York et al., Carb. Res. 285:99-128, 1996; Liang et al., Science 274:1520-1522, 1996; Ding et al., Adv. Expt. Med. Biol. 376:261-269, 1995); a lipoprotein library (de Kruif et al., FEBS Lett. 3 99:23 2-23 6, 1996); a glycoprotein or glycolipid library (Karaoglu et al., J Cell Biol. 130.567-577, 1995); or a chemical library containing, for example, drugs or other pharmaceutical agents (Gordon et al., J Med. Chem. 37.1385-1401, 1994; Ecker and Crooke, BioTechnology 13:351-360, 1995). Polynucleotides can be particularly useful as agents that can alter a function of stem cells or progenitor cells because nucleic acid molecules having binding specificity for cellular targets, including cellular polypeptides, exist naturally, and because synthetic molecules having such specificity can be readily prepared and identified (see, for example, U.S. Pat. No. 5,750,342).

In one embodiment, for a high throughput format, MSCs can be introduced into wells of a multiwell plate or of a glass slide or microchip, and can be contacted with the test agent. Generally, the cells are organized in an array, particularly an addressable array, such that robotics conveniently can be used for manipulating the cells and solutions and for monitoring the MSCs, particularly with respect to the function being examined. An advantage of using a high throughput format is that a number of test agents can be examined in parallel, and, if desired, control reactions also can be run under identical conditions as the test conditions. As such, the methods disclosed herein provide a means to screen one, a few, or a large number of test agents in order to identify an agent that can alter a function of MSCs, for example, an agent that induces the MSCs to differentiate into a desired cell type, or that prevents spontaneous differentiation, for example, by maintaining a high level of expression of regulatory molecules.

The cells are contacted with test compounds sufficient for the compound to interact with the cell. When the compound binds a discrete receptor, the cells are contacted for a sufficient time for the agent to bind its receptor. In some embodiments, the cells are incubated with the test compound for an amount of time sufficient to affect phosphorylation of a substrate. In some embodiments, cells are treated in vitro with test compounds at 37° C. in a 5% CO₂ humidified atmosphere. Following treatment with test compounds, cells are washed with Ca²+ and Mg²+ free PBS and total protein is extracted as described (Haldar et al., Cell Death Diff. 1:109-115, 1994; Haldar et al., Nature 342:195-198, 1989; Haldar et al., Cancer Res. 54:2095-2097, 1994). In additional embodiments, serial dilutions of test compound are used.

### EXAMPLES

The disclosure is illustrated by the following non-limiting Examples.

### Example 1

### AAVS-copGFP Donor Vector Construction and AAVS TALEN mRNAs generation

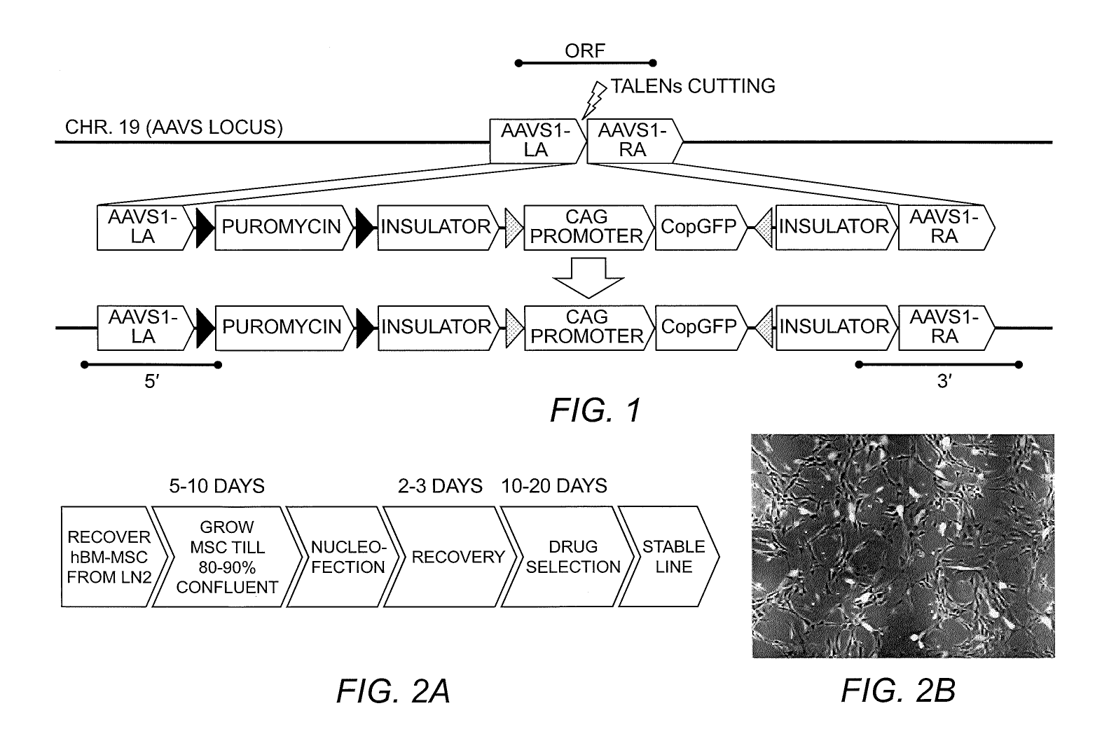

A backbone vector containing a puromycin resistant gene flanked by two loxP sites and a CAG promoter driving copGFP cassette was constructed between the lox2272 and lox511 sites. Two insulators were inserted in the AAVS1-copGFP donor vector targeting to the AAVS1 site at Chr.19 (see FIG. 1). A 754 bp left homologous arm and an 838 bp right homologous arm were amplified by PCR from XCL1 (Xcell Inc, CA) gDNA and cloned into the backbone vector. TALEN expression plasmids targeting AAVS safe harbor site in Chr.19 were provided by NIH. Each plasmid DNA was linearized by XbaI for mRNA production and purification following modified manufacturer's protocols.

### Example 2

### Generation of an MSC Line Stably Expressing AAVS-copGFP

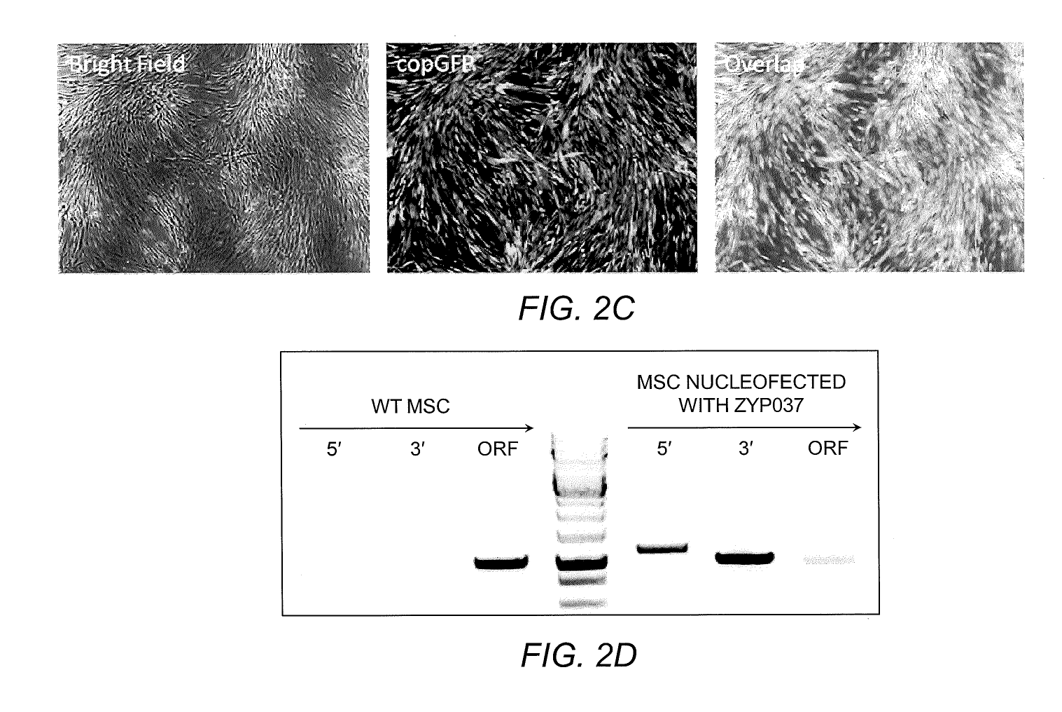

Human Bone Marrow-Derived Mesenchymal Stem Cells (MSC) were purchased from RoosterBio Inc. (Frederick, Md.) and cell recovery and maintaining was performed in accordance with the manufacturer's protocol. In brief, cells were grown in MSC High Performance Media (RoosterBio Inc., MD) in T-25 flasks and media were changed every 3-4 days. On the day of nucleofection, cells should be about 80-90% confluent and are in monolayer. 0.05% Trypsin-EDTA (Life Tech., NJ) was used to generate single cell suspension. After three times PBS washes, 2×10⁶ MSC were nucleofected with 4-6 μg of each AAVS TALEN RNAs together with 5 μg donor vector (AAVS-copGFP) using Amaxa Human Stem Cell Nucleofection Kit (Lonza, N.J.) and were plated in a new T-25 flask with fresh MSC High Performance Media. The process is summarized in FIG. 2A.

As shown in FIG. 2B, the nucleofection efficiency was about 60% and at this stage, all green cells contained episomal vectors. After nucleofection, MSC were recovered for 2-3 days to reach 80-90% confluent before puromycin selection. A stable puromycin resistant cell population was obtained two weeks after drug selection. More than 98% of the cell population was green fluorescent (FIG. 2C). Junction PCR were also performed to confirm the successful integration of AAVS-copGFP construct into MSC line (FIG. 2D). Both 5′ and 3′ arm PCR bands indicating correct homologous recombination, which was observed in the stable AAVS-copGFP MSC line but not in the WT MSC linea ORF bands were detected in both WT and AAVS-copGFP MSC indicating that there are mixed population, both heterozygotes and homozygotes, in the transfected line (FIG. 2D).

The stable line was expanded and cryopreserved using a freezing solution which is MSC High Performance Media containing 10% DMSO.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

1. A method for introducing a polynucleotide of interest into a safe harbor locus in a genome of a mesenchymal stem cell, comprising
introducing into the mesenchymal stem cell (a) an upstream transcription activator-like effector nuclease (TALEN) comprising an upstream DNA-binding domain linked to a DNA cleavage domain, wherein the upstream DNA binding domain specifically binds to the safe-harbor locus at a site upstream of a genomic insertion site in the genome of the mesenchymal stem cell, (b) a downstream transcription activator-like effector nuclease (TALEN) comprising a downstream DNA-binding domain linked to a DNA cleavage domain, wherein the downstream DNA binding domain specifically binds to the safe-harbor locus at a site downstream of the genomic insertion site in the genome of the mesenchymal stem cell, and (c) a single or double-stranded donor polynucleotide comprising sense and/or antisense strand polynucleotide overhangs that are complementary to corresponding polynucleotide overhangs of cleaved the genomic DNA when cleaved at the genomic insertion site, wherein the complementary overhangs facilitate homologous recombination of the donor polynucleotide with the cleaved genomic DNA,
thereby introducing the donor polynucleotide into the genomic insertion site into the safe harbor locus in the genome of the mesenchymal stem cell.

2. The method of claim 1, wherein the upstream TALEN binds to the sense strand of a genomic DNA locus flanking the insertion site and the downstream TALEN binds to the antisense strand of a genomic DNA locus flanking the insertion site.

3-5. (canceled)

6. The method of claim 1, wherein the donor polynucleotide encodes an agent for inducing the proliferation and/or differentiation of the mesenchymal stem cells into selected mature cells and/or tissues selected from the group consisting of adipocytes, cartilage, bone, tendons, muscle, skin, myocytes, neurons and glia; the donor polynucleotide encodes a selectable marker and/or a detectable label; or the donor polynucleotide comprises a lineage specific promoter operably linked to a selectable marker and/or detectable label.

7-9. (canceled)

10. The method of claim 1, wherein the safe harbor locus is AAVS1 or CYBL.

11-12. (canceled)

13. The method of claim 1, wherein the downstream TALEN comprises SEQ ID NO:11; the DNA cleavage domain comprises a FokI nuclease domain; the genomic sense strand locus bound by the upstream TALEN comprises SEQ ID NO: 1; the genomic antisense strand locus bound by the downstream TALEN comprises SEQ ID NO: 3; the upstream TALEN comprises the amino acid sequence set forth as SEQ ID NO: 7, SEQ ID NO:8 or SEQ ID NO:10; or the downstream TALEN comprises the sequence of SEQ ID NO: 11.

14. (canceled)

15. The method of claim 13, wherein the FokI nuclease domain comprises SEQ ID NO: 13.

16-20. (canceled)

21. The method of claim 1, wherein the cell comprises two copies of each chromosome, and wherein the polynucleotide is inserted into the two copies of the same chromosome.

22. A method of modifying the genomic DNA of a mesenchymal stem cell, comprising introducing into the cell (a) an upstream transcription activator-like effector nuclease (TALEN) comprising an upstream DNA-binding domain linked to a DNA cleavage domain, wherein the upstream DNA binding domain specifically binds to a site upstream of a genomic sequence of interest, and (b) a downstream transcription activator-like effector nuclease (TALEN) comprising a downstream DNA-binding domain linked to a DNA cleavage domain, wherein the downstream DNA binding domain specifically binds to a site downstream of a genomic sequence of interest, whereby the transcription activator-like effector nucleases cleave the genomic DNA and excise the genomic sequence of interest, thereby modifying the genomic DNA of the cell.

23. (canceled)

24. The method of claim 22, wherein the upstream TALEN comprises SEQ ID NO:8; the downstream TALEN comprises SEQ ID NO:11; the DNA cleavage domain comprises a FokI nuclease; the genomic sense strand locus bound by the upstream TALEN comprises SEQ ID NO: 1; or the genomic antisense strand locus bound by the downstream TALEN comprises SEQ ID NO: 3.

25-26. (canceled)

27. The method of claim 24, wherein the DNA cleavage domain derived from a FokI nuclease comprises SEQ ID NO: 13.

28. The method of claim 22, wherein the upstream TALEN binds to the sense strand of a genomic DNA locus flanking the sequence of interest and the downstream TALEN binds to the antisense strand of a genomic DNA locus flanking the sequence of interest.

29. The method of claim 22, wherein the genomic sequence of interest is in the AAVS1 safe harbor locus or the CYBL safe harbor locus.

30-34. (canceled)

35. A method of assessing the physiological effect of a polypeptide on a mesenchymal stem cell comprising
introducing into the mesenchymal stem cell in accordance with claim 1 a polynucleotide encoding the polypeptide of interest; and
assessing a parameter of the mesenchymal stem cell, thereby determining the physiological effect of the polypeptide on the mesenchymal stem cell.

36. The method of claim 35, wherein the parameter is division of the mesenchymal stem cell or differentiation of the mesenchymal stem cell.

37. (canceled)

38. A method for inducing a mesenchymal stem cell to differentiate into a selected mature cell or tissue, said method comprising introducing into the mesenchymal stem cell (a) an upstream transcription activator-like effector nuclease (TALEN) comprising an upstream DNA-binding domain linked to a DNA cleavage domain, wherein the upstream DNA binding domain specifically binds to the safe-harbor locus at a site upstream of a genomic insertion site in the genome of the mesenchymal stem cell, (b) a downstream transcription activator-like effector nuclease (TALEN) comprising a downstream DNA-binding domain linked to a DNA cleavage domain, wherein the downstream DNA binding domain specifically binds to the safe-harbor locus at a site downstream of the genomic insertion site in the genome of the MSC, and (c) a single or double-stranded donor polynucleotide comprising sense and/or antisense strand polynucleotide overhangs that are complementary to corresponding polynucleotide overhangs of cleaved the genomic DNA when cleaved at the genomic insertion site, wherein the donor polynucleotide encodes one or more factors sufficient to differentiate the mesenchymal stem cell into the selected mature cell or tissue.

39. (canceled)

40. A mesenchymal stem cell modified in accordance with the method of 22.

41. A method for treating a selected disease or disorder in a subject, said method comprising administering to the subject a therapeutically effective amount of the mesenchymal stem cells of claim 40.

42. The method of the claim 41 wherein the disease or disorder is an inflammatory or immune, a neurological, a cancer, or a cardiovascular disease or disorder.

43. The method of claim 41 wherein the disease or disorder relates to absence of a protein.

44. (canceled)

45. The method of claim 41 wherein the mesenchymal stem cell produces an antibody useful in treating a disease or disorder wherein antibody treatment is warranted.


SEQUENCE LISTING (21 sequences)

SEQ ID NO: 1 - DNA, 17 bp, HUMAN
cttacccttctcccatt

SEQ ID NO: 2 - DNA, 1692 bp, HUMAN
ctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaa

SEQ ID NO: 3 - DNA, 16 bp, HUMAN
cccaaaatatatttat

SEQ ID NO: 4 - DNA, 1590 bp, HUMAN
ctgaccccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaa

SEQ ID NO: 5 - DNA, 2889 bp, HUMAN
gtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggccttgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccctgaacctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccatctggtggcgttggcatgtcttggtggacgtcccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagatct

SEQ ID NO: 6 - DNA, 2787 bp, HUMAN
gtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggccttgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccctgaacctgaccccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccatctggtggcgttggcatgtcttggtggacgtcccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagatct

SEQ ID NO: 7 - protein, 564 aa, HUMAN
LTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALE

SEQ ID NO: 8 - protein, 963 aa, HUMAN
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASNGGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 9 - DNA, 3006 bp, HUMAN
atggactacaaagaccatgacggtgattataaagatcatgacatcgattacaaggatgacgatgacaagatggcccccaagaagaagaggaaggtgggcatccacggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggccttgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccctgaacctgaccccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccatctggtggcgttggcatgtcttggtggacgtcccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagatct

SEQ ID NO: 10 - protein, 530 aa, HUMAN
LTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALE

SEQ ID NO: 11 - protein, 929 aa, HUMAN
VDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPDQVVAIASHDGGKQALETVQRLLPVLCQDHGLTPDQVVAIASHDGGKQALETVQRLLPVLCQDHGLTPDQVVAIASHDGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNGGGKQALETVQRLLPVLCQDHGLTPDQVVAIASNIGGKQALETVQRLLPVLCQDHGLTPDQVVAIASNGGGKQALESIVAQLSRPDPALAALTNDHLVALACLGGRPALDAVKKGLPHAPALIKRTNRRIPERTSHRVAGSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 12 - DNA, 2904 bp, HUMAN
atggactacaaagaccatgacggtgattataaagatcatgacatcgattacaaggatgacgatgacaagatggcccccaagaagaagaggaaggtgggcatccacggggtacctatggtggacttgaggacactcggttattcgcaacagcaacaggagaaaatcaagcctaaggtcaggagcaccgtcgcgcaacaccacgaggcgcttgtggggcatggcttcactcatgcgcatattgtcgcgctttcacagcaccctgcggcgcttgggacggtggctgtcaaataccaagatatgattgcggccctgcccgaagccacgcacgaggcaattgtaggggtcggtaaacagtggtcgggagcgcgagcacttgaggccttgctgactgtggcgggtgagcttagggggcctccgctccagctcgacaccgggcagctgctgaagatcgcgaagagagggggagtaacagcggtagaggcagtgcacgcctggcgcaatgcgctcaccggggcccccctgaacctgaccccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgactccggaccaagtggtggctatcgccagccacgatggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacggtggcggcaagcaagcgctcgaaacggtgcagcggctgttgccggtgctgtgccaggaccatggcctgaccccggaccaagtggtggctatcgccagcaacattggcggcaagcaagcgctcgaaacggtgcagcggctgttgccggtgctgtgccaggaccatggcctgaccccggaccaagtggtggctatcgccagcaacggtggcggcaagcaagcgctcgaaagcattgtggcccagctgagccggcctgatccggcgttggccgcgttgaccaacgaccatctggtggcgttggcatgtcttggtggacgtcccgcgctcgatgcagtcaaaaagggtctgcctcatgctcccgcattgatcaaaagaaccaaccggcggattcccgagagaacttcccatcgagtcgcgggatcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagatct

SEQ ID NO: 13 - protein, 199 aa, HUMAN
SQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFRS

SEQ ID NO: 14 - DNA, 597 bp, HUMAN
tcccaactagtcaaaagtgaactggaggagaagaaatctgaacttcgtcataaattgaaatatgtgcctcatgaatatattgaattaattgaaattgccagaaattccactcaggatagaattcttgaaatgaaggtaatggaattttttatgaaagtttatggatatagaggtaaacatttgggtggatcaaggaaaccggacggagcaatttatactgtcggatctcctattgattacggtgtgatcgtggatactaaagcttatagcggaggttataatctgccaattggccaagcagatgaaatgcaacgatatgtcgaagaaaatcaaacacgaaacaaacatatcaaccctaatgaatggtggaaagtctatccatcttctgtaacggaatttaagtttttatttgtgagtggtcactttaaaggaaactacaaagctcagcttacacgattaaatcatatcactaattgtaatggagctgttcttagtgtagaagagcttttaattggtggagaaatgattaaagccggcacattaaccttagaggaagtcagacggaaatttaataacggcgagataaactttagatct

SEQ ID NO: 15 - DNA, 21 bp, HUMAN
cccaagaagaagaggaaggtg

SEQ ID NO: 16 - protein, 7 aa, HUMAN
PKKKRKV

SEQ ID NO: 17 - DNA, 69 bp, HUMAN
atggactacaaagaccatgacggtgattataaagatcatgacatcgattacaaggatgacgatgacaag

SEQ ID NO: 18 - protein, 23 aa, HUMAN
MDYKDHDGDYKDHDIDYKDDDDK

SEQ ID NO: 19 - DNA, 49 bp, HUMAN
cttacccttctcccatttcctcatccttccaacataaatatattttggg

SEQ ID NO: 20 - DNA, 20 bp, HUMAN
ctgccgtctctctcctgagt

SEQ ID NO: 21 - DNA, 20 bp, HUMAN
gtgggcttgtactcggtcat